SEQUENCE LISTING (6 sequences)

SEQ ID NO: 1 - DNA, 20 bp, synthetic construct
  source 1..20: mol_type = other DNA
  misc_difference 2: note = locked nucleic acid (LNA)
  misc_difference 5: note = locked nucleic acid (LNA)
  misc_difference 8: note = locked nucleic acid (LNA)
  misc_difference 13: note = locked nucleic acid (LNA)
  misc_difference 16: note = locked nucleic acid (LNA)
  misc_difference 19: note = locked nucleic acid (LNA)
cagcagcagcagcagcagca

SEQ ID NO: 2 - protein, 14 aa, synthetic construct
  SITE 5: note = bAla
  SITE 11: note = bAla
  SITE 14: note = bAla
  SITE 2: note = 6-aminohexanoic acid
  SITE 8: note = 6-aminohexanoic acid
  SITE 13: note = 6-aminohexanoic acid
RXRRBRRXRRBRXB

SEQ ID NO: 3 - DNA, 27 bp, Mus musculus
  source 1..27: mol_type = other DNA
caatcattactgatcgaattgaaagaa

SEQ ID NO: 4 - DNA, 32 bp, Mus musculus
  source 1..32: mol_type = other DNA
agaaaccaagcagttggccaaagacctccgtc

SEQ ID NO: 5 - DNA, 51 bp, synthetic construct
  source 1..51: mol_type = other DNA
aagctagaacaatcattactgatcgaacagttggccaaagacctccgtcaa

SEQ ID NO: 6 - DNA, 37 bp, synthetic construct
  source 1..37: mol_type = other DNA
aatgatcatcaaacagaagcagttggccaaagacctc

(19) United States
(12) Patent Application Publication
(10) Pub. No.: US 2025/0163418 A1
(43) Pub. Date: May 22, 2025

(54) Brush polymer-oligonucleotide conjugates for the treatment of muscular dystrophy

(71) Applicant: Northeastern University, Boston, MA (US)

(72) Inventor: Ke Zhang, Natick, MA (US)

(21) Appl. No.: 18/940,823

(22) Filed: Nov. 7, 2024

Related U.S. Application Data
(60) Provisional application No. 63/596,859, filed on Nov. 7, 2023.

(51) Int. Cl. C12N 15/113; A61P 21/00

(52) U.S. Cl. CPC C12N 15/113; A61P 21/00; C12N 2310/14; C12N 2310/3231; C12N 2310/351

(57) ABSTRACT

Provided herein are, in various embodiments, methods and compositions for treating muscular dystrophy in a subject in need thereof, comprising administering to the subject a therapeutically effective amount of a composition comprising a bottlebrush polymer-oligonucleotide conjugate. Also provided herein are methods of making bottlebrush polymer-oligonucleotide conjugates.

### RELATED APPLICATIONS

This application claims the benefit of U.S. Provisional Application No. 63/596,859, filed Nov. 7, 2023. The entire teachings of the above application are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 52002389001_SL.xml; created Feb. 10, 2025, 10,973 Bytes in size.

### GOVERNMENT SUPPORT

This invention was made with government support under Grant No. 1R01GM121612 awarded by the National Institutes of Health. The government has certain rights in the invention.

### BACKGROUND

Muscular dystrophy comprises a group of genetic disorders characterized by progressive weakening of muscles and wasting of muscle tissue. Muscular dystrophy affects about 1 in 5,000 people, yet there remains no approved curative treatment. One explanation for this is the inability of nucleic acid drugs to effectively infiltrate muscle tissues following intravenous injection. Additionally, muscular dystrophies such as myotonic dystrophy type 1 (DM1) necessitate sufficient drug localization to the nucleus, where pathogenic mutant RNA is trapped, adding another hurdle to overcome after endocytosis.

### SUMMARY

In one aspect, the disclosure provides a method for treating muscular dystrophy in a subject in need thereof, comprising administering to the subject a therapeutically effective amount of a composition comprising a bottlebrush (i.e., brush) polymer-oligonucleotide conjugate.

In some embodiments, the subject has muscular dystrophy selected from Duchenne muscular dystrophy (DMD), myotonic dystrophy type 1 (DM1), and facioscapulohumeral muscular dystrophy (FSHD).

In some embodiments, the bottlebrush polymer-oligonucleotide conjugate comprises: a polymer backbone; polyethylene glycol (PEG) polymer arms covalently linked to the polymer backbone; and an oligonucleotide covalently linked to the backbone.

In some embodiments, the oligonucleotide comprises: a single stranded nucleic acid; a double stranded nucleic acid; a chemically modified nucleic acid; or any combination of the foregoing.

In some embodiments, the oligonucleotide comprises: one or more locked nucleic acid (LNA) modified bases; an antisense oligonucleotide (ASO); a small interfering RNA (siRNA); a sequence complementary to a region of a pathogenic transcript; or any combination of the foregoing.

In some embodiments, the pathogenic transcript comprises: a human DM1 protein kinase (DMPK) transcript containing CUG repeats; a mutated human dystrophin (DMD) transcript; or a human double homeobox 4 (DUX4) transcript.

In some embodiments, the oligonucleotide comprises a nucleic acid sequence of 5′-CAGCAGCAG-3′.

In some embodiments, the polymer backbone comprises one or more monomers.

In some embodiments, at least one of the monomers is selected from a synthetic monomer, a natural monomer, and a modified form of a natural molecule.

In some embodiments, the synthetic monomer is selected from serinol, norbornene, acrylate, and acrylamide; the natural monomer is selected from amino acid and sugar; the modified form of a natural molecule is selected from a morpholino phosphorodiamidate, a modified amino acid, a modified spermine, a modified lipid, and a modified cholesterol; or any combination of the foregoing.

In some embodiments, the bottlebrush polymer-oligonucleotide conjugate comprises about 15 to about 30 PEG polymer arms; each of the PEG polymer arms is about 2 kDa to about 20 kDa; or both of the foregoing.

In some embodiments, the bottlebrush polymer-oligonucleotide conjugate comprises about 30 PEG polymer arms; each of the PEG polymer arms is about 10 kDa; or both of the foregoing.

In some embodiments, the bottlebrush polymer-oligonucleotide conjugate further comprises a radiochemical label, a targeting ligand, or both.

In some embodiments, the targeting ligand comprises a nuclear localization sequence (NLS) peptide, a transferrin receptor 1-targeting peptide, or a pH-sensitive lipid.

In some embodiments, administering the bottlebrush polymer-oligonucleotide conjugate to the subject induces alternative splicing.

In some embodiments, the composition is administered to the subject: as a dose of about 1 mg oligonucleotide per kg bodyweight (1 mg/kg) to about 80 mg oligonucleotide per kg bodyweight (80 mg/kg); by intravenous injection, intramuscular injection, intraperitoneal injection, or subcutaneous injection; or any combination of the foregoing.

In another aspect, the disclosure provides a bottlebrush polymer-oligonucleotide conjugate, comprising: a polymer backbone; polyethylene glycol (PEG) polymer arms covalently linked to the polymer backbone; and an oligonucleotide covalently linked to the backbone, wherein the oligonucleotide comprises a sequence complementary to a region of a pathogenic transcript associated with a muscular dystrophy.

In another aspect, the disclosure provides a composition comprising the bottlebrush polymer-oligonucleotide conjugate, wherein the bottlebrush polymer-oligonucleotide conjugate comprises a polymer backbone; polyethylene glycol (PEG) polymer arms covalently linked to the polymer backbone; and an oligonucleotide covalently linked to the backbone, wherein the oligonucleotide comprises a sequence complementary to a region of a pathogenic transcript associated with a muscular dystrophy.

In another aspect, the disclosure provides a method of producing a bottlebrush polymer-oligonucleotide conjugate comprising: synthesizing a bottlebrush polymer; and conjugating the bottlebrush polymer with an oligonucleotide comprising a sequence complementary to a region of a pathogenic transcript associated with a muscular dystrophy, thereby producing the bottlebrush polymer-oligonucleotide conjugate.

In some embodiments, synthesizing the bottlebrush polymer comprises polymerizing norbornenyl bromide and norbornenyl PEG; and conjugating the bottlebrush polymer with the oligonucleotide comprises: conjugating the bottlebrush polymer with an azide group, thereby producing an azide-functionalized bottlebrush polymer; and reacting the azide-functionalized bottlebrush polymer and the oligonucleotide, wherein the oligonucleotide is a DBCO-modified oligonucleotide, thereby producing the bottlebrush polymer-oligonucleotide conjugate.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 illustrates the interaction between dystrophin and actin filaments.

FIGS. 2A-2E. pacDNA outperformed B-peptide conjugates without the associated toxicity. FIG. 2A shows Sanger sequencing chromatograms showing exon 53 inclusion (top; SEQ ID NOs:3-4) and therapeutic exon 53 skipping (bottom; SEQ ID NO:5). FIG. 2B shows reverse-transcription polymerase chain reaction (RT-PCR) results showing relative amounts of native dystrophin and exon 53-skipped dystrophin in differentiated myotubes upon treatment with various concentrations of pacDNA, a bottlebrush polymer-oligonucleotide conjugate (also known as BRUSHIELD®-DNA). GAPDH is shown as a housekeeping gene. C2C12 cells were used to generate myotubes. FIG. 2C illustrates a mouse model study comprising repeated dosing of B-peptide conjugate or pacDNA. FIG. 2D shows results from gel electrophoresis of cDNA showing relative amounts of native dystrophin and exon 53-skipped dystrophin in various tissues of a mouse model upon treatment with various concentrations of pacDNA or B-peptide conjugate. FIG. 2E is a graph that quantifies the efficiency of exon 53 skipping in various mouse tissues as shown in FIG. BD. Pec, pectoral; TB, triceps brachii; Quad, quadriceps muscle; Gas, gastrocnemius; TA, tibialis anterior; Dia, diaphragm.

FIGS. 3A-3C. pacDNA enabled dual-exon skipping in vivo. FIG. 3A illustrates that multiple oligonucleotides (e.g., antisense oligonucleotides (ASOs)) can be conjugated to the same bottlebrush polymer, which enables simultaneous delivery to the same cell. FIG. 3B shows RT-PCR and Sanger sequencing results confirming presence of exon 53 skipping in C2C12 myotubes after treatment with pacDNA (SEQ ID NO:6). FIG. 3C shows RT-PCR results confirming the presence of dual-exon skipping 1 week after treatment of an mdx4cv mouse with a single intravenous (i.v.) dose of pacDNA (15 mg/kg ASO1 and 15 mg/kg ASO2). Percent efficiency in exon skipping is shown below the treatment lanes.

FIGS. 4A-4B. pacDNA can provide favorable systemic delivery and mechanism of action (MOA) for myotonic dystrophy type 1 (DM1). FIG. 4A illustrates DM1 pathogenesis. Muscle blind-like 1 (MBNL) RNA-binding protein forms heterogeneous nuclear ribonucleoproteins (hnRNPs) with DM1 protein kinase (DMPK) pre-mRNA containing a CUG trinucleotide repeat expansion (r(CUG)ₙ), leading to spliceopathy. FIG. 4B illustrates that the unique protein-shielding mechanism of pacDNA bottlebrush polymer-oligonucleotide conjugate is ideal for DM1 therapy.

FIGS. 5A-5F. pacDNA synthesis, characterization, and muscle biodistribution. FIG. 5A is a synthesis scheme of pacDNA via ring-opening metathesis polymerization (ROMP) with custom oligonucleotide “click” chemistry conjugation. The inset illustrates a pacDNA comprising a polymer backbone (tan), polyethylene glycol (PEG) arms covalently bound to the polymer backbone, and an oligonucleotide (e.g., an ASO) covalently bound to the polymer backbone. FIG. 5B shows results from size exclusion chromatography (SEC) performed on the pacDNA. Mn, number average molecular weight; Mw, weight average molecular weight; PDI, polydispersity index. FIG. 5C is a transmission electron microscopy (TEM) image assessing uniformity and morphology of the pacDNA. FIGS. 5D, 5E, and 5F quantify ex vivo biodistribution of ⁸⁹Zr-labeled pacDNA and ⁸⁹Zr-DNA as percentage of the injected dose per gram of tissue (% ID/gram) measured over 14 days (n=4 per time point) in CD-1® mice. FIGS. 5D and 5E show results in several different tissues; FIG. 5F compares results from heart and muscle tissues.

FIGS. 6A-6C. PacDNA effect on CUG^{exp} foci. FIG. 6A is a summary quantification of the number of CUG^{exp} foci per nucleus in nontreated (NT) or 1 μM L9-pacDNA treated fibroblasts. About 150-200 nuclei were counted per sample. FIG. 6B shows fluorescence in situ hybridization (FISH) of primary human DM1 fibroblasts (GM03989). Left panel shows DAPI staining of nuclei (blue), right panel showing only nuclear foci (yellow). Scale bar=20 μm. FIG. 6C shows distribution of Cy5-tagged L9-pacDNA (green) in human fibroblasts. Scale bar=50 μm.

FIGS. 7A-7C. pacDNA showed promise for DM1 therapy. HSA-LR20b (HSA-LR or HSA^{LR}) mice were given a single 40 nanomole (nmol) i.v. dose (40 nmol ASO in 6 mg/kg L9-pacDNA or L9-B-peptide) via the tail vein; mice were sacrificed 14 days post injection. Data is shown as fold changes relative to wild-type (FVB/N) mouse. ASO is a full locked nucleic acid (LNA) sequence. FIG. 7A is a graph quantifying fold change in SLN expression. FIG. 7B is a graph quantifying fold change in MYO1A expression. FIG. 7C is a graph quantifying fold change in UCHL1 expression.

FIGS. 8A-8C. DM1 Splicing Panel. FIGS. 8A and 8B depict percent spliced-in (PSI) of 18 DM1-associated splicing events in wild-type (WT) mice (n=5), nontreated HSA-LR20b (NT) mice (n=2), and HSA-LR20b mice treated with one (1×; n=2), four (4×; n=2), and eight doses (8×; n=2) of L9-pacDNA. Each dose was 5.3 mg bottlebrush polymer-ASO conjugate per kg body weight (mg/kg) (40 nanomoles, ASO basis (i.e., a dose of bottlebrush polymer-ASO conjugate comprising 40 nanomoles of ASO)), and quadricep muscles were collected 2 weeks after the final injection. FIG. 8C quantifies additional splicing events. The y-axis of all plots in FIGS. 8A-8C shows PSI.

FIGS. 9A-9F. Global splicing correction following L9-pacDNA treatment. FIG. 9A quantifies percent corrected to wild-type inclusion levels across 18-gene fragment panel of DM1-associated splice events (FIGS. 8A, 8B) (Tukey box and whisker plot) (**p<0.01). The value above each box is that group's average percent correction across the panel of 18 splicing events. FIGS. 9B, 9C, and 9D are percent spliced-in (PSI) graphs for DM1-associated splice events in HSA-LR20b mice treated with unconjugated bottlebrush polymer (Brush; n=2) and L9^{scr}-pacDNA (Scramble; n=2). PSI for wild type (WT) and nontreated (NT) mice are shown for comparison. FIG. 9E is a splicing correction summary of mice treated with four doses of L9-pacDNA (L9P 4×) across all significant (FDR<0.05) dysregulated splice events in HSA-LR20b mice vs. wild type. Percent values represent the extent to which treatment shifted exon inclusion back to wild-type levels. FIG. 9F is a stacked bar chart showing the percentage recovered after 4×L9-pacDNA for significantly altered splicing events in each splicing mode (recovered defined as FDR>0.01 vs WT).

FIGS. 10A-10E. Gene set enrichment analysis in pacDNA-treated mice. FIG. 10A shows select GSEA categories of genes corrected (FDR>0.01 vs. WT) in mice treated with four doses of L9-pacDNA (L9P 4×). The value next to each bar is the adjusted p-value of that category. FIG. 10B plots significant (padj<0.05) biological processes (BP, n=42) downregulated in nontreated (NT) and L9-pacDNA-treated (L9P 1×, L9P 4×, L9P 8×) HSA-LR20b mouse quadriceps vs. FVB/n wild-type (WT) mice (log₂ fc<−1, padj<0.05). FIG. 10C shows significantly downregulated (pink) and upregulated (green) cellular components (CC) in L9P 8× treated mice vs. nontreated HSA-LR20b mice. FIG. 10D shows significantly downregulated (pink) and upregulated (green) Kyoto Encyclopedia of Genes and Genomes (KEGG) pathways in L9P 8× treated mice vs. nontreated HSA-LR20b mice. FIG. 10E shows significantly upregulated (green) biological processes (BP) in L9P 8× treated mice vs. nontreated HSA-LR20b mice. Numbers next to all bars in FIGS. 10C-10E correspond to the number of genes detected in that gene set.

FIGS. 11A-11B. CAG repeat DNA. FIG. 11A shows correlation of relative repeat RNA levels with alternative splicing in DM1-related splice events. Datapoints represent 15 total samples from nontreated mice (NT, n=2), mice treated with one dose (1×; n=2), four doses (4×; n=2), and eight doses (8×; n=3) of L9-pacDNA (L9P), one dose of L9-B-peptide (L9-Bpep; n=2), one dose of L9-free (L9; n=2), one dose of L9^{scr}-pacDNA (Scramble; n=1), and free brush polymer (Brush; n=1). FIG. 11B shows relative levels of CAG repeat DNA in each sample, generated from CUG repeat RNA. Samples were normalized to the average of two HSA-LR20b samples (relative (CUG)ₙ RNA level=1).

FIG. 12. Expression of CUG-repeat RNA. FIG. 12 shows expression levels of CUG-repeat-containing RNA from endogenous genes containing short (2-25) CTG repeats in mice treated with one, four, or eight doses of L9-pacDNA (L9P 1×, L9P 4×, and L9P 8×, respectively; see Table 3) compared to expression in nontreated (NT) mice. Log₂ FCvsNT, log₂ fold-change in expression in treated vs. untreated mice.

FIGS. 13A-13E. Functional effects of L9-pacDNA treatment in HSA-LR20b mice. FIG. 13A illustrates a regimen in which the HSA-LR20b mice received intravenous injections of L9-pacDNA (10.6 mg/kg/dose) for 4 consecutive days in the first week, and then once weekly over 12 additional weeks. FIG. 13B quantifies myotonia scores that were measured weekly. The response to each pinch was classified as severe myotonia (>1 s, 3), myotonia (0.5-1 s, 2), quick recovery myotonia (<0.5 s, 1), single leg myotonia (0.5), or no myotonia (0). FIG. 13C shows body weight measurements that were recorded throughout the administration period in FIG. 13A. Right panel shows separate data for male and female mice. FIG. 13D is a grip strength analysis that demonstrates improved muscle strength in the treated group compared to the non-treated group at week 12 (**p<0.01). The mean and standard error of the mean(SEM) (n=10 per group) in wild-type (WT), nontreated HSA-LR20b (NT), and treated 10.6 mg/kg/dose HSA-LR20b are displayed. FIG. 13E shows grip strength over time. **p<0.01, ***p<0.001, ****p<0.0001 by one-way ANOVA followed by Tukey multiple comparison correction.

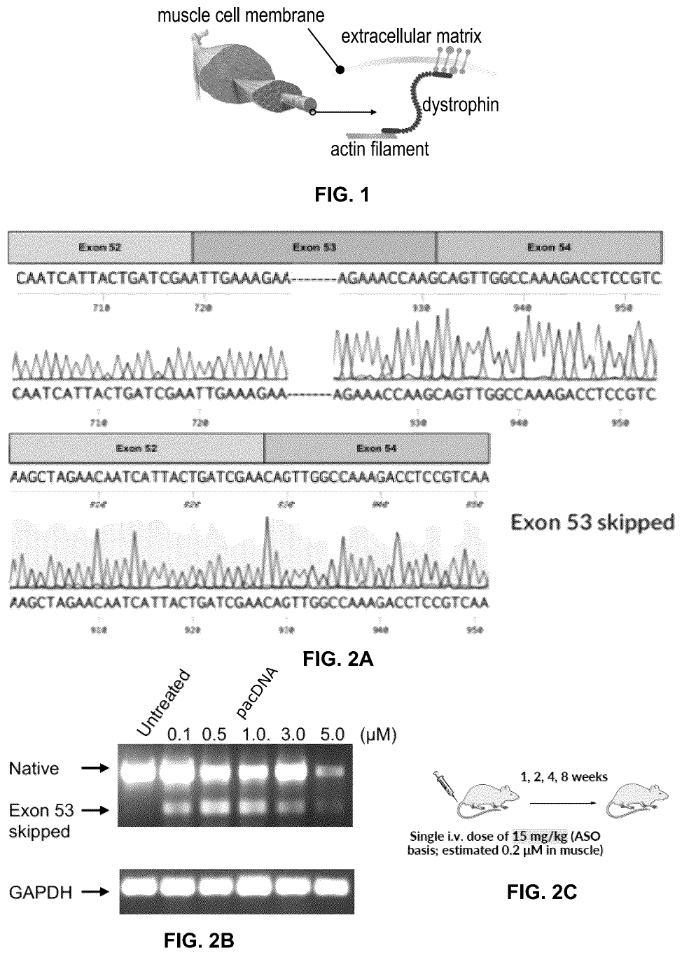

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

### INTRODUCTION

Conjugation of oligonucleotides to polyethylene glycol-based brush polymers (termed polymer-assisted compaction of DNA, or pacDNA) has been shown to limit their off-target interactions, improve pharmacokinetics, and increase tissue accumulation without interfering with target hybridization. The findings disclosed herein show that a pacDNA conjugate targeting mutant CUG trinucleotide repeat expansion (CUG^{exp}) RNA tracts in a mouse model of myotonic dystrophy type 1 (DM1) achieved partial correction of DM1-associated alternative splicing at low nanomole doses. These mice also exhibited improvements in myotonia, body weight, and grip strength with repeated dosing. With properties of enhanced muscle bioavailability, blood half-life, and endonuclease stability, the pacDNA technology bridges the gaps present for traditional antisense drugs, representing a potentially more potent, durable, and cost-effective DM1 therapy.

Myotonic dystrophy type 1 (DM1) is a multisystemic muscle-wasting disease caused by an unstable CTG repeat expansion mutation in the 3′ untranslated region (UTR) of the DM1 protein kinase (DMPK) gene. DM1 affects 1 in 2,100 individuals and is the most common form of adult-onset muscular dystrophy, affecting not only the muscles but the eyes, central nervous system, skin, and cardiac, pulmonary, gastrointestinal, endocrine, and reproductive systems as well.¹ The autosomal dominant mutation lengthens in successive generations, leading to worsened disease severity and younger symptom onset.² Pathogenesis is primarily attributed to the loss-of-function (e.g., the loss of availability via entrapment) of muscle blind-like 1 (MBNL1) RNA-binding protein, a regulator of nuclear pre-mRNA splicing (FIG. 4A).³ MBNL1 colocalizes with DMPK CUG-repeat RNA hairpins, forming large nuclear aggregates or foci, leading to altered signaling pathways and gene splicing abnormalities.³⁻⁵ Loss of DMPK alone does not result in major features of DM1, and thus transgenic mouse models with long (CTG)ₙ gene insertions (e.g., HSA-LR20b, with a (CTG)₂₂₀ human ACTA1 transgene) have been developed to study DM1.⁶

Nucleic acid therapeutics are a growing class of drugs with attractive qualities, including high specificity, customizability, and robust synthesis pipelines. Single-stranded nucleic acid polymers, known as antisense oligonucleotides (ASOs), exert their therapeutic effect by hybridizing to and blocking or enzymatically degrading their target RNA. Recent commercial advances in exon-skipping therapeutics for Duchenne muscular dystrophy (DMD) and spinal muscular atrophy (SMA) highlight the promise of ASOs for muscular dystrophies.⁷⁻⁸ In DM1, targeting CUG-repeat RNA can prevent secondary structure formation and subsequent entrapment of splicing proteins (e.g., MBNL1), leading to a recovery in the widespread alternative splicing responsible for the DM1 phenotype.⁹⁻¹¹. The magnitude of CUG-repeat RNA has been directly related to disease severity, and both small interfering RNA (siRNA) and blocking ASOs have been reported to achieve significant repeat RNA reduction accompanied by partial splicing correction.¹¹⁻¹³

Traditional phosphodiester backbone ASO efficacy in muscle is limited by poor biodistribution to these tissues, rapid renal clearance, and low plasma protein binding and membrane permeability.¹⁴⁻¹⁷ Bypassing these issues by increasing dosing not only raises costs but can introduce non-specific drug toxicity and immunogenicity.¹¹⁻²¹ Various modifications on the ribose moiety (2′-O-methyl, 2′ to 4′ methylene bridges (locked nucleic acid (LNA)), or phosphorodiamidate morpholino oligomer (PMO)) or phosphorothioate (PS) linkage chemistry can increase ASO endonuclease resistance and bolster RNA/ASO duplex stability, and have recently been leveraged in preclinical candidates.²²⁻²⁶ Yet, there remains no approved curative drug for DM1, with existing treatments focused on symptom management.²⁷ Several delivery strategies have been deployed to increase ASO muscle biodistribution, endocytosis, and nuclear trafficking, including conjugation with cationic cell-penetrating peptides (CPP) and transferrin-receptor 1 targeting antibodies.²⁸⁻³⁰ While showing potential in early clinical stages, these delivery vehicles may elicit nonspecific immune responses, cause hematopoietic cell toxicity, and result in membrane perturbation and leakage.³¹⁻³⁴

Conjugation of oligonucleotides to branched polyethylene glycol (PEG) bottlebrush polymers with high molecular weights and entropic shielding capabilities can limit their off-target interactions and immunogenicity.^{35,36} While structurally simple, these bottlebrush polymer-oligonucleotide conjugates, termed pacDNA (polymer-assisted compaction of DNA) or BRUSHIELD®-DNA evade renal clearance due to their large size (about 300 kDa, about 20 nm), improve plasma pharmacokinetics (25-fold and 19-fold increase in elimination half-life and area under the curve, respectively vs. free siRNA)³⁵, and enhance cell uptake by promoting membrane adsorption and macropinocytosis.³⁷⁻³⁹ These structures shield the targeting ASO from DNA-protein interactions without interfering with target hybridization kinetics (FIG. 4C).⁴⁰ By avoiding opsonization and other DNA-protein interactions, the pacDNA eludes phagocytic clearance, limits anti-carrier adaptive immunity, and reduces all side effects that derive from these interactions, making it uniquely suitable for long-term applications. The polymer protects the ASO from premature extracellular cleavage and may greatly prolong its intracellular lifetime. The results of a study testing a pacDNA drug for steric blocking of toxic CUG-repeat RNA in myotonic dystrophy type 1 mouse skeletal muscles is described herein.

The development of the first ASO therapy for DM1, Baliforsen (also known as IONIS 598769 or ISIS-DMPKRx; Ionis Pharmaceuticals, Inc., Carlsbad, CA, USA), was discontinued in 2017 citing insufficient drug concentrations in muscle after subcutaneous injection.⁶⁰ Moreover, the systemic pathogenesis of DM1 renders certain drug administration modalities, such as intramuscular injection, ineffective or impractical for total symptom addressment. The current landscape of DM1 therapies in development includes several candidates that attempt to boost the pharmacokinetic properties of intravenous (i.v.) dosed nucleic acid therapies, using cell-penetrating peptide, monoclonal antibody (mAb), or fragment antigen binding (Fab) conjugation for enhanced delivery.^{61,62} Avidity Biosciences (San Diego, CA, USA) and Dyne Therapeutics (Waltham, MA, USA) are in early clinical trials testing a transferrin receptor 1 targeting mAb-siRNA conjugate (AOC-1001) or Fab-ASO conjugate (Dyne-101) for degradation of mutant DMPK RNA.^{63,64} Data released for these compounds shows clinical improvements in several functional DM1 tests and relatively low occurrences of adverse events. However, muscle biopsies from patients in the high-dose cohorts of Dyne-101 and AOC-1001, given 5.4 mg/kg or 8 mg/kg respectively, displayed just 19% and 12% mean correction of mis-splicing in their respective panels, indicating significant room for improvement. While complete correction of alternative splicing may not be necessary, further improvement could translate to meaningful clinical benefits in patients.

The pacDNA technology disclosed herein pioneered a long-lasting, safe, and non-immunogenic drug that vastly and reliably exceeded the previous therapeutic threshold of splicing correction of 12-19%. In some embodiments, the pacDNA may be dosed intravenously (i.v.) as infrequently as once every few months. It is biologically inert, and showed low antigenicity and interactions with metabolic proteins, removing the risk of drug-induced toxicity and coagulopathy. Since the pacDNA is composed mainly of polyethylene glycol, it costs far less to produce than other delivery vehicles such as peptides, monoclonal antibodies, or viral vectors, increasing the likelihood that DM1 patients' insurance could subsidize costs.

Another advantage of the pacDNA technology is its potential for iterative optimization with functional alterations to the brush polymer, through either polymer backbone modifications or attachment of targeting ligands that can improve pharmacokinetic (PK) properties, assist with cell/nuclear uptake, or facilitate endosomal escape. In some embodiments, these additions allow the pacDNA structure to evolve alongside the understanding of what drives cell and nuclear uptake, intracellular stability, and therapeutic efficiency. Initial screenings on modified monomers (spermine, lipid, cholesterol) indicate that the backbone chemistry of the polymer and modifier arrangement within the backbone has strong effects (100-fold difference in range) on cell uptake through energy-dependent macropinocytosis and scavenger receptor type A-dependent mechanisms.³⁷ Other targeting ligands such as nuclear localization sequence (NLS) peptides, transferrin receptor 1 targeting peptides, or pH-sensitive lipids may also be attached to the polymer.⁶⁵⁻⁷⁰

The study described herein served as a basis for this fundamentally new ASO delivery technology for the systemic treatment of muscle-based RNA diseases, development in DM1, myotonic dystrophy type 2 (DM2), Duchenne muscular dystrophy (DMD), Facioscapulohumeral muscular dystrophy (FSHD), and other diseases amenable to ASO targeting RNA in the muscle.

### Abbreviations

3′-UTR: Three prime untranslated region
ASO: Antisense oligonucleotide
BP: Biological processes
CLCN1: Chloride voltage-gated channel 1
CPG: Controlled-pore glass
CPP: Cell-penetrating peptide
CUGBP1: CUG binding protein 1
DBCO: Dibenzocyclooctyne
DFO: Desferrioxamine
DLS: Dynamic light scattering
DM1: Myotonic dystrophy type 1
DM2: Myotonic dystrophy type 2
DMD: Duchenne muscular dystrophy
DMPK: DM1 Protein Kinase
DMT: Dimethoxytrityl
DIPEA: N,N-diisopropylethylamine
EDCI: 1-Ethyl-3-(3-dimethylaminopropyl)carbodiimide
ELISA: Enzyme-linked immunosorbent assay
FDR: False discovery rate
FISH: Fluorescence in situ hybridization
FPLC: Fast protein liquid chromatography
FSHD: Facioscapulohumeral muscular dystrophy
GPC: Gel permeation chromatography
GSEA: Gene set enrichment analysis
I.V.: Intravenous
LNA: Locked nucleic acid
MBNL1: Muscle blind-like one
pacDNA: Polymer-assisted compaction of DNA
PEG: Polyethylene glycol
PMO: Phosphorodiamidate morpholino oligomer
PS: Phosphorothioate
PSI: Percent spliced in
rMATS: R multivariate analysis of transcript splicing
RP-HPLC: Reversed-phase high-performance liquid chromatography
SLC8A3: Solute carrier family 8 member A3
SMA: Spinal muscular atrophy
⁸⁹Zr: Zirconium-89

### Example Features of Polymer-ASO Conjugates

In some embodiments, the disclosure provides bottlebrush polymer-oligonucleotide (e.g., pacDNA or BRUSHIELD®-DNA) conjugate therapeutics for myotonic dystrophy type 1 (DM1). Bottlebrush-oligonucleotide conjugates have not been tested in muscular dystrophy models prior to the present disclosure. In some embodiments, the conjugate exhibits one or more of the following attributes: enhanced plasma pharmacokinetics (PK), e.g., muscle bioavailability, blood half-life, and endonuclease stability; better distribution into muscle and other tissues (estimated 500× higher than free oligonucleotides); target engagement in the muscle; longer drug action in the muscle compared with a peptide-conjugated phosphorodiamidate morpholino oligomer (PPMO); none of the toxicities observed for PPMO.

In some embodiments, the conjugate requires reduced dosing and dosing frequency for DMD oligonucleotide drugs. In some embodiments, the conjugate exhibits increased potency compared to other DMD or DM1 therapies.

Current DMD therapeutics are ineffective, high in cost of goods (COGS), and require weekly dosing. The conjugate disclosed herein can solve these difficulties. In some embodiments, the disclosed conjugate has the potential to generate first-in-class therapies for DMD and other muscular diseases.

While a study disclosed herein involves Duchenne muscular dystrophy models, the disclosed bottlebrush-oligonucleotide conjugates can be applicable in other genetic muscular diseases such as facioscapulohumeral muscular dystrophy (FSHD) and myotonic muscular dystrophy (e.g., DM1).

In some embodiments of the disclosure, the compositions and methods provide for a method of treating a disease or disorder, comprising administering to a subject in need thereof, a therapeutically effective amount of a bottlebrush polymer-oligonucleotide conjugate. The terms “bottlebrush” and “brush” are used interchangeably herein. As used herein, the terms “polymer-assisted compaction of DNA” (pacDNA) and “BRUSHIELD®-DNA” or “Brushield™-DNA” refer to a bottlebrush polymer-oligonucleotide conjugate, wherein the oligonucleotide comprises a nucleic acid, e.g., DNA or RNA. As used herein, the term “BRUSHIELD®” or “Brushield™” is used to refer to the bottlebrush polymer portion of a bottlebrush polymer-oligonucleotide conjugate. In some instances, which will be clear to a person of skill in the art by context, “BRUSHIELD®” or “Brushield™” may also be used to refer to BRUSHIELD®-DNA.

In some embodiments, a method for treating muscular dystrophy in a subject in need thereof comprises administering to the subject a therapeutically effective amount of a composition comprising a bottlebrush polymer-oligonucleotide conjugate.

As used herein, the term “muscular dystrophy” refers to a group of genetic disorders that are characterized by progressive weakening of muscles and wasting of muscle tissue. Examples of types of muscular dystrophy include Emery-Dreifuss, limb-girdle (dysferlinopathy, Erb's), pelvifemoral, scapulohumeral, facioscapulohumeral (Landouzy-Dejerine), late-onset distal, myotonic, oculopharyngeal, scapuloperoneal, congenital, early onset distal, Becker (benign pseudohypertrophic), and Duchenne (pseudohypertrophic) muscular dystrophies. In some embodiments, a muscular dystrophy is a dystrophinopathy, i.e., associated with a pathogenic change to the dystrophin gene (DMD) and/or gene product (e.g., a pathogenic deletion or mutation). Dystrophinopathies include, e.g., Duchenne muscular dystrophy or Becker muscular dystrophy. Examples of myotonic dystrophy include myotonic dystrophy type 1 (DM1) and myotonic dystrophy type 2 (DM2).

As used herein, “therapy,” “treat,” “treating,” or “treatment” means inhibiting or relieving a condition in a subject in need thereof. For example, a therapy or treatment refers to any of. (i) the prevention of symptoms associated with a disease or disorder (e.g., muscular dystrophy); (ii) the postponement of development of the symptoms associated with a disease or disorder (e.g., muscular dystrophy); and/or (iii) the reduction in the severity of such symptoms that will, or are expected, to develop with said disease or disorder (e.g., muscular dystrophy). The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment. In some embodiments, the symptoms comprise one or mor of the symptoms described herein. In some embodiments, the symptom is improvement in myotonia, body weight, and/or grip strength.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy (therapeutically effective) or treatment. The effectiveness of a therapy or treatment (e.g., eliciting a humoral and/or cellular immune response) can be determined by suitable methods known in the art.

In some embodiments, the subject has muscular dystrophy selected from Duchenne muscular dystrophy (DMD), myotonic dystrophy type 1 (DM1), and facioscapulohumeral muscular dystrophy (FSHD). In some embodiments, the subject has Duchenne muscular dystrophy. In some embodiments, the subject has myotonic dystrophy type 1 (DM1). In some embodiments, the subject has facioscapulohumeral muscular dystrophy (FSHD).

In some embodiments, the subject has a muscular dystrophy mediated by RNA, e.g., a CUG trinucleotide repeat expansion (CUG^{exp}) or a mutant transcript (e.g., a transcript that leads to a protein with loss of function or reduced function). In some embodiments, a CUG^{exp} is associated with aberrant RNA-binding protein (RBP) activity (e.g., loss of wild-type activity), aberrant splicing (i.e., mis-splicing), or both.

The terms “subject” and “patient” are used interchangeably herein. The term “patient” refers to a human, while the term “subject” may refer to a human or a non-human animal. As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a non-human mammal is an FVB/N mouse (Friend Virus B NIH Jackson, Strain #001800). In some embodiments, a non-human mammal is an mdx^{4Cv} mouse (model of Duchenne muscular dystrophy (DMD); homozygous for the Rag1^{null} and X-linked Dmd^{mdc-4Cv} and IL2rγ^{null} mutations (males hemizygous for X-linked mutations)). In some embodiments, a subject is a transgenic non-human mammal, e.g., a non-human mammal carrying a human gene. In some embodiments, a transgenic non-human mammal is an HSA-LR20b mouse (human skeletal actin long repeat line 20b, Strain #032031; abbreviated as HSA-LR or HSA^{LR}). In some embodiments, a subject is a human. In still further embodiments, a subject of the disclosure may be a cell, cell culture, tissue, organ, or organ system.

In some embodiments the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years.

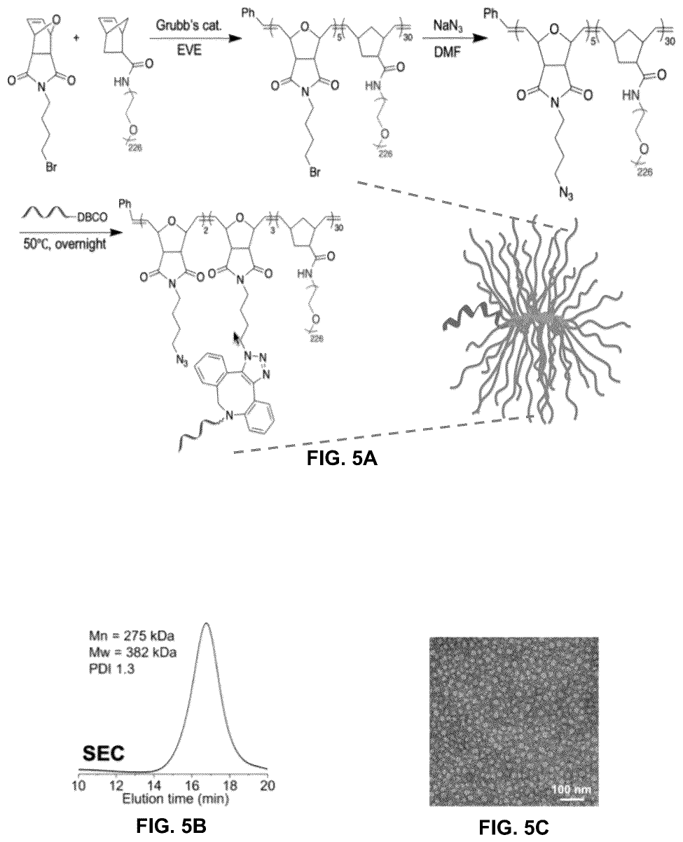

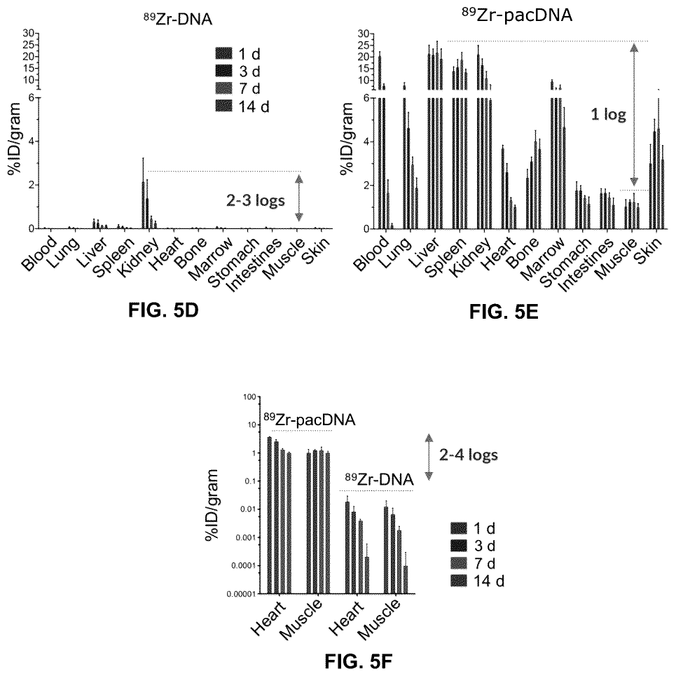

In some embodiments, a bottlebrush polymer-oligonucleotide conjugate comprises: a polymer backbone; polyethylene glycol (PEG) polymer arms covalently linked to the polymer backbone; and an oligonucleotide covalently linked to the backbone. In some embodiments, a bottlebrush polymer-oligonucleotide conjugate comprises a bottlebrush polymer, wherein the bottlebrush polymer comprises a polymer backbone and polyethylene glycol (PEG) polymer arms covalently linked to the polymer backbone. See FIG. 5A, inset, for an illustration of an example embodiment of a bottlebrush polymer-oligonucleotide conjugate.

In one aspect of the disclosure, there is provided a bottlebrush polymer-oligonucleotide conjugate comprising a polymer backbone, wherein the polymer backbone comprises a plurality of monomers. Each individual monomer can be covalently attached to an oligonucleotide or a polyethylene glycol (PEG) arm (i.e., one oligonucleotide per monomer, or one PEG arm per monomer). At least two of the plurality of monomers is covalently attached to a polyethylene glycol (PEG) arm (i.e., one PEG arm is attached to one monomer). At least two of the plurality of monomers is covalently attached to a polyethylene glycol (PEG) arm (i.e., one PEG arm is attached to one monomer).

In some embodiments, every monomer of the polymer backbone need not be covalently attached to an oligonucleotide or a polyethylene glycol (PEG) arm. In some embodiments, a monomer is attached to a PEG arm or derivatized with a functional group prior to being assembled into a polymer backbone. In some embodiments, a functionally derivatized monomer within a backbone is conjugated to an oligonucleotide.

The PEG arms and the at least one oligonucleotide can be attached to the polymer backbone in any order. For example, in some embodiments, a terminal monomer of the backbone is covalently linked to an oligonucleotide. In some embodiments, a terminal monomer of the backbone is covalently linked to a PEG arm. In some embodiments, an internal monomer of the backbone is covalently linked to an oligonucleotide. In some embodiments, an internal monomer of the backbone is covalently linked to a PEG arm.

In one aspect of the disclosure, there is provided a bottlebrush polymer-oligonucleotide conjugate comprising: a polymer backbone; polyethylene glycol (PEG) polymer arms covalently linked to the polymer backbone; and at least one oligonucleotide covalently linked to the backbone. In some embodiments, a bottlebrush polymer-oligonucleotide conjugate comprises two or more oligonucleotides. In some embodiments, a bottlebrush polymer-oligonucleotide conjugate comprises three or more oligonucleotides. In some embodiments, a bottlebrush polymer-oligonucleotide conjugate comprises one oligonucleotide. In some embodiments, a bottlebrush polymer-oligonucleotide conjugate comprises two oligonucleotides. In some embodiments, a bottlebrush polymer-oligonucleotide conjugate comprises three oligonucleotides. In some embodiments, a bottlebrush polymer-oligonucleotide conjugate comprises one to about three oligonucleotides. In some embodiments, a bottlebrush polymer-oligonucleotide conjugate comprises two or more oligonucleotides, wherein each of the oligonucleotides comprise the same sequence. In some embodiments, a bottlebrush polymer-oligonucleotide conjugate comprises two or more oligonucleotides, wherein each of the oligonucleotides comprise different sequences.

In some embodiments, an oligonucleotide (i.e., the oligonucleotide of a bottlebrush polymer-oligonucleotide conjugate) comprises a single stranded nucleic acid, a double stranded nucleic acid, a chemically modified nucleic acid, or any combination of the foregoing. In some embodiments, an oligonucleotide comprises a single stranded nucleic acid. In some embodiments, an oligonucleotide comprises a double stranded nucleic acid. In some embodiments, an oligonucleotide comprises a chemically modified nucleic acid. In some embodiments, an oligonucleotide comprises a chemically modified single stranded nucleic acid. In some embodiments, an oligonucleotide comprises a chemically modified double stranded nucleic acid.

In some embodiments, an oligonucleotide (i.e., the oligonucleotide of a bottlebrush polymer-oligonucleotide conjugate) has a length of about 8 bases or base pairs to about 35 bases or base pairs. In some embodiments, an oligonucleotide has a length of about 9 bases or base pairs, about 26 bases or base pairs, or about 31 bases or base pairs. In some embodiments, an oligonucleotide has a length of about 9 bases or base pairs. In some embodiments, an oligonucleotide has a length of less than about 50 bases or base pairs.

In some embodiments, an oligonucleotide (i.e., the oligonucleotide of a bottlebrush polymer-oligonucleotide conjugate) comprises one or more locked nucleic acid (LNA) modified bases, an antisense oligonucleotide (ASO), a small interfering RNA (siRNA), a sequence complementary to a region of a pathogenic transcript, or any combination of the foregoing.

In some embodiments, an oligonucleotide comprises one or more locked nucleic acid (LNA) modified bases. In some embodiments, an oligonucleotide comprises all locked nucleic acid (LNA) modified bases. In some embodiments, an oligonucleotide comprises one or more stabilizing modifications, functionalizing modifications, or both. As used herein the term “locked nucleic acid” (LNA), also known as bridged nucleic acid (BNA), is a synthetic nucleic acid analog wherein the ribose ring is conformationally locked by a methylene bridge connecting the 2′-O to the 4′C.

In some embodiments, an oligonucleotide comprises a small interfering RNA (siRNA). In some embodiments, an oligonucleotide comprises an antisense oligonucleotide (ASO). In some embodiments, the ASO comprises single stranded DNA or single stranded RNA; a chemically modified ASO or an unmodified ASO; or a combination of the foregoing. In some embodiments, the ASO comprises single stranded DNA or single stranded RNA. In some embodiments, the ASO comprises single stranded DNA. In some embodiments, the ASO comprises single stranded RNA. In some embodiments, the ASO comprises a chemically modified ASO or an unmodified ASO. In some embodiments, the ASO comprises unmodified ASO. In some embodiments, the ASO comprises locked nucleic acid (LNA) modified bases. In some embodiments, the ASO comprises a chemically modified ASO.

Examples of chemical modifications that can be applied to oligonucleotides (e.g., ASOs) to produce chemically modified oligonucleotides include, but are not limited to: 2′ to 4′ methylene bridges (i.e., locked nucleic acid (LNA)); functionalization with DBCO (5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite)); a fluorescent dye (e.g., cyanine 3 (Cy3) or cyanine 5 (Cy5)); 2′-O-methylation; 2′-O-alkyl modifications; phosphorodiamidate morpholino oligomer (PMO); phosphorothioate (PS) linkage chemistry; and 4′-thio modifications. The aforementioned modifications and other modifications are well-known to those of skill in the art.

In some embodiments, an oligonucleotide comprises a sequence complementary to a region of a pathogenic transcript. As used herein, “region of a transcript,” e.g., a pathogenic transcript, means a portion of the transcript, e.g., a portion of the transcript to which a sequence of an oligonucleotide of a bottlebrush polymer-oligonucleotide conjugate is complementary. A region of a transcript may be a linear sequence, e.g., a CUG trinucleotide repeat, or a non-linear structure, such as a hairpin. As expressed herein, an oligonucleotide comprising a sequence complementary to a region of a pathogenic transcript is understood to “target” the pathogenic transcript. The term “pathogenic” refers to an agent that causes a disease or that is likely or suspected to cause a disease.

In some embodiments, a pathogenic transcript comprises a human DM1 protein kinase (DMPK) transcript containing CUG repeats, a mutated human dystrophin (DMD) transcript, or a human double homeobox 4 (DUX4) transcript. In some embodiments, a pathogenic transcript comprises a human DM1 protein kinase (DMPK) transcript containing CUG repeats. In some embodiments, a pathogenic transcript comprises a human DM1 protein kinase (DMPK) transcript. In some embodiments, a pathogenic transcript comprises a human DM1 protein kinase (DMPK) transcript containing a CUG repeat expansion, i.e., a number of CUG repeats that is greater than the number of CUG repeats found in non-pathogenic DMPK transcripts. In some embodiments, a pathogenic transcript comprises a mutated human dystrophin (DMD) transcript. In some embodiments, a mutated human dystrophin (DMD) transcript comprises a loss-of-function mutation, e.g., a deletion, an insertion, a duplication, or a point mutation. In some embodiments, a pathogenic transcript comprises a human double homeobox 4 (DUX4) transcript. In some embodiments, a DUX4 transcript is aberrantly expressed, e.g., through loss of epigenetic silencing.

In some embodiments, the subject has Duchenne muscular dystrophy and a pathogenic transcript comprises a mutated human dystrophin (DMD) transcript. In some embodiments, the subject has myotonic dystrophy type 1 (DM1) and a pathogenic transcript comprises a human DM1 protein kinase (DMPK) transcript containing CUG repeats. In some embodiments, the subject has facioscapulohumeral muscular dystrophy (FSHD) and a pathogenic transcript comprises a human double homeobox 4 (DUX4) transcript.

In some embodiments, an oligonucleotide comprises a nucleic acid sequence that targets a human DM1 protein kinase (DMPK) transcript containing CUG repeats, a mutated human dystrophin (DMD) transcript, or a human double homeobox 4 (DUX4) transcript. In some embodiments, an oligonucleotide comprises a nucleic acid sequence that targets a human DM1 protein kinase (DMPK) transcript containing a CUG repeat expansion, i.e., a number of CUG repeats that is greater than the number of CUG repeats found in non-pathogenic DMPK transcripts. In some embodiments, an oligonucleotide comprises a nucleic acid sequence of 5′-(CAG)ₙ-3′, wherein “n” is about 3 (e.g., 2 to 4) to about 5 (e.g., 4 to 6). In some embodiments, an oligonucleotide comprises a nucleic acid sequence of 5′-CAGCAGCAG-3′.

In some embodiments, the ASO targets a CUG trinucleotide repeat (i.e., a CUG repeat, a CUG repeat expansion). In some embodiments, the ASO comprises a sequence that targets a CUG repeat. In some embodiments, the ASO comprises a sequence that is complementary to a CUG repeat. In some embodiments, the ASO comprises a sequence of 5′-(CAG)ₙ-3′, wherein “n” is about 3 (e.g., 2 to 4) to about 5 (e.g., 4 to 6). In some embodiments, the ASO comprises a sequence of 5′-CAGCAGCAG-3′.

In some embodiments, a polymer backbone of a bottlebrush polymer-oligonucleotide conjugate comprises one or more monomers. In some embodiments, at least one of the monomers is selected from a synthetic monomer, a natural monomer, and a modified form of a natural molecule. In some embodiments, at least one of the monomers is a synthetic monomer. In some embodiments, at least one of the monomers is a natural monomer. In some embodiments, at least one of the monomers is a modified form of a natural molecule. In some embodiments, at least one of the monomers is a synthetic monomer and at least one of the monomers is a natural monomer. In some embodiments, at least one of the monomers is a synthetic monomer and at least one of the monomers is a modified form of a natural molecule. In some embodiments, at least one of the monomers is a natural monomer and at least one of the monomers is a modified form of a natural molecule. In some embodiments, at least one of the monomers is a synthetic monomer, at least one of the monomers is a natural monomer, and at least one of the monomers is a modified form of a natural molecule. In some embodiments, a polymer backbone comprises a combination of one or more of a synthetic monomer, a natural monomer, and a modified form of a natural molecule.

In some embodiments, the synthetic monomer is selected from serinol, norbornene, acrylate, and acrylamide; the natural monomer is selected from amino acid and sugar; the modified form of a natural molecule is selected from a morpholino phosphorodiamidate, a modified amino acid, a modified spermine, a modified lipid, and a modified cholesterol; or any combination of the foregoing. In some embodiments, a synthetic monomer is selected from serinol, norbornene, acrylate, and acrylamide. In some embodiments, a synthetic monomer is serinol. In some embodiments, a synthetic monomer is norbornene. In some embodiments, a synthetic monomer is acrylate. In some embodiments, a synthetic monomer is acrylamide. In some embodiments, a natural monomer is selected from amino acid and sugar. In some embodiments, a natural monomer is an amino acid. In some embodiments, a natural monomer is a sugar. In some embodiments, a modified form of a natural molecule is selected from a morpholino phosphorodiamidate, a modified amino acid, a modified spermine, a modified lipid, and a modified cholesterol. In some embodiments, a modified form of a natural molecule is a morpholino phosphorodiamidate. In some embodiments, a modified form of a natural molecule is a modified amino acid. In some embodiments, a modified form of a natural molecule is a modified spermine. In some embodiments, a modified form of a natural molecule is a modified lipid. In some embodiments, a modified form of a natural molecule is a modified cholesterol.

In some embodiments, the backbone comprises one or more nanoparticles or monomers. In some embodiments, the backbone comprises one or more nanoparticles. An example of a nanoparticle is a fullerene C₆₀. In some embodiments, the monomers are selected from synthetic monomers, natural monomers, or modified forms of natural molecules (e.g., natural monomers).

In some embodiments, a bottlebrush polymer-oligonucleotide conjugate comprises a diblock polymer, i.e., a copolymer comprising two different monomers. An example of two different monomers includes norbornenyl bromide (or a derivative thereof, e.g., norbornenyl azide or norbornenyl conjugated to an oligonucleotide (i.e., norbornenyl-oligonucleotide) and norbornenyl PEG. As used herein, the term “copolymer” means a polymer produced by addition polymerization between two or more different monomers. In some embodiments, a bottlebrush polymer-oligonucleotide conjugate comprises a triblock polymer, i.e., a copolymer comprising three different monomers.

In some embodiments, the bottlebrush polymer-oligonucleotide conjugate comprises about 15 to about 40 monomers. In some embodiments, the bottlebrush polymer-oligonucleotide conjugate comprises about 35 monomers. In some embodiments, the bottlebrush polymer-oligonucleotide conjugate comprises about 5-30 monomers. In some embodiments, the bottlebrush polymer-oligonucleotide conjugate comprises about 15 to about 30 norbornenyl PEG monomers. In some embodiments, the bottlebrush polymer-oligonucleotide conjugate comprises about 30 norbornenyl PEG monomers. In some embodiments, the bottlebrush polymer-oligonucleotide conjugate comprises about 30 norbornenyl PEG monomers and about 5 norbornenyl bromide monomers or derivatives of norbornenyl bromide monomers (e.g., norbornenyl azide or norbornenyl-oligonucleotide). In some embodiments, the bottlebrush polymer-oligonucleotide conjugate comprises about 30 norbornenyl PEG monomers and about 5 derivatives of norbornenyl bromide monomers (e.g., about 2 norbornenyl azide monomers and about 3 norbornenyl-oligonucleotide monomers).

In some embodiments, the bottlebrush polymer-oligonucleotide conjugate has a high molecular weight. In some embodiments, the bottlebrush polymer-oligonucleotide conjugate has a molecular weight of about 275 kDa to about 382 kDa. In some embodiments, the bottlebrush polymer-oligonucleotide conjugate has a molecular weight of about 275 kDa. In some embodiments, the bottlebrush polymer-oligonucleotide conjugate has a molecular weight of about 382 kDa. In some embodiments, the bottlebrush polymer-oligonucleotide conjugate has a molecular weight of about 275,000 g/mol to about 382,000 g/mol. In some embodiments, the bottlebrush polymer-oligonucleotide conjugate has a molecular weight of about 275,000 g/mol. In some embodiments, the bottlebrush polymer-oligonucleotide conjugate has a molecular weight of about 382,000 g/mol. In some embodiments, the bottlebrush polymer-oligonucleotide conjugate has a molecular weight of about 303,027 g/mol.

In some embodiments, the bottlebrush polymer-oligonucleotide conjugate comprises about 15 to about 30 PEG polymer arms; each of the PEG polymer arms is about 2 kDa to about 20 kDa; or both of the foregoing. In some embodiments, the bottlebrush polymer-oligonucleotide conjugate comprises about 15 to about 30 PEG polymer arms. In some embodiments, the bottlebrush polymer-oligonucleotide conjugate comprises about 15 PEG polymer arms. In some embodiments, the bottlebrush polymer-oligonucleotide conjugate comprises about 30 PEG polymer arms. In some embodiments, each of the PEG polymer arms is about 2 kDa to about 20 kDa.

In some embodiments, the bottlebrush polymer-oligonucleotide conjugate comprises about 30 PEG polymer arms; each of the PEG polymer arms is about 10 kDa; or both of the foregoing. In some embodiments, the bottlebrush polymer-oligonucleotide conjugate comprises about 30 PEG polymer arms. In some embodiments, each of the PEG polymer arms is about 10 kDa.

In some embodiments, each of the PEG polymer arms comprises 10 kDa PEG (i.e., PEG with a molecular weight of about 10 kDa). In some embodiments, each of the PEG polymer arms comprises about 226 ethylene glycol units.

In some embodiments, the bottlebrush polymer-oligonucleotide conjugate exhibits a spherical morphology.

In some embodiments, the bottlebrush polymer-oligonucleotide conjugate further comprises a radiochemical label, a targeting ligand, or both. In some embodiments, the bottlebrush polymer-oligonucleotide conjugate further comprises a radiochemical label. In some embodiments, the bottlebrush polymer-oligonucleotide conjugate further comprises a targeting ligand. In some embodiments, the bottlebrush polymer-oligonucleotide conjugate further comprises a radiochemical label and a targeting ligand. In some embodiments, a targeting ligand comprises a nuclear localization sequence (NLS) peptide, a transferrin receptor 1-targeting peptide, or a pH-sensitive lipid. In some embodiments, a targeting ligand comprises a nuclear localization sequence (NLS) peptide. In some embodiments, a targeting ligand comprises a transferrin receptor 1-targeting peptide. In some embodiments, a targeting ligand comprises a pH-sensitive lipid.

In some embodiments, administering a bottlebrush polymer-oligonucleotide conjugate to a subject induces alternative splicing. In some embodiments, administering a bottlebrush polymer-oligonucleotide conjugate to a subject induces exon skipping. In some embodiments, an effect of administering a bottlebrush polymer-oligonucleotide conjugate to a subject is determined by the nucleic acid sequence of the oligonucleotide.

In another aspect, the disclosure provides methods for inducing alternative splicing in a subject in need thereof, the method comprising administering a bottlebrush polymer-oligonucleotide conjugate to the subject. In another aspect, the disclosure provides methods for improving muscle tone in a subject in need thereof, the method comprising administering a bottlebrush polymer-oligonucleotide conjugate to the subject.

In another aspect, the disclosure provides methods for improving grip strength in a subject in need thereof, the method comprising administering a bottlebrush polymer-oligonucleotide conjugate to the subject.

In another aspect, the disclosure provides methods for reducing muscle weakness and/or muscle wasting in a subject in need thereof, the method comprising administering a bottlebrush polymer-oligonucleotide conjugate to the subject.

In some embodiments, administering the bottlebrush polymer-oligonucleotide conjugate to the subject induces splicing correction. In some embodiments, administering the bottlebrush polymer-oligonucleotide conjugate to the subject induces global splicing correction. As used herein, “splicing correction” refers to reverting an aberrant splicing (mis-splicing) pattern or a pathogenic splicing pattern to a wild-type splicing pattern or a non-pathogenic splicing pattern.

In some aspects, a composition, e.g., a composition a bottlebrush polymer-oligonucleotide conjugate, is provided herein.

In some embodiments, the composition is administered to the subject: as a dose of about 1 mg oligonucleotide per kg bodyweight (1 mg/kg) to about 80 mg oligonucleotide per kg bodyweight (80 mg/kg); by intravenous injection, intramuscular injection, intraperitoneal injection, or subcutaneous injection; or any combination of the foregoing.

In some embodiments, the composition is administered to the subject: as a dose of about 5 mg oligonucleotide per kg bodyweight (5 mg/kg) to about 11 mg oligonucleotide per kg bodyweight (11 mg/kg). In some embodiments, the composition is administered to the subject: as a dose of about 5.3 mg oligonucleotide per kg bodyweight (5.3 mg/kg) to about 10.6 mg oligonucleotide per kg bodyweight (10.6 mg/kg). In some embodiments, the composition is administered to the subject at a dose of about 5.4 mg/kg-8 mg/kg.

In some embodiments, a composition comprising a bottlebrush polymer-oligonucleotide conjugate is administered to the subject: once a week for twelve consecutive weeks; once a day for four consecutive days, then once a week for twelve consecutive weeks; once a month for at least two consecutive months; or once every two to three months for at least two consecutive cycles. In some embodiments, a composition may be dosed intravenously (i.v.) every one month, two months or three months.

In some embodiments, the composition is administered to the subject by injection, aerosol inhalation, infusion, ingestion, or a combination thereof. In some embodiments, the composition is administered to the subject by injection. In some embodiments, the composition is administered to the subject by intravenous injection, intramuscular injection, or subcutaneous injection. In some embodiments, the composition is administered to the subject by intravenous (i.v.) injection.

Determining the dosage and route of administration for a particular agent, patient and disease or condition is well within the abilities of one of skill in the art. In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection. In some embodiments the route of administration is intramuscular, intranodal, intravenous, intradermal, subcutaneous, intranasal, infusion, intraperitoneal, intracranial, intratracheal or epicardial. Preferably, the dosage does not cause or produces minimal adverse side effects. In some embodiments, the conjugate is administered with phosphate-buffered saline (PBS).

In some embodiments the route of administration is determined by the tissue or tissues, or organ to which the agent or agents are targeted. In some embodiments, the tissue, tissues, or organ is the subject's lung, ovary, immune system, skin, blood vessel, muscle, blood, brain, heart, intestine(s), pancreas, spleen, kidney, heart, bone, bone marrow, stomach, head, or any combination thereof.

In another aspect, the disclosure provides a composition that comprises a bottlebrush polymer-oligonucleotide conjugate. In some embodiments, the compositions and methods further comprise a pharmaceutically acceptable carrier. As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

In some embodiments, compositions as described herein are used in combination with other known agents (e.g., additional therapeutic agents) and therapies, which are used for treatment of muscular dystrophy. In some embodiments, compositions as described herein are used in combination with other known agents (e.g., additional therapeutic agents) and therapies, such as chemotherapy, transplantation, and radiotherapy. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, different treatments (e.g., additional therapeutics) can be administered simultaneously or sequentially.

In still further embodiments, the method comprises administering to the subject an effective amount of the composition, or a pharmaceutically acceptable salt thereof.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in subjects.

In some embodiments, the pharmaceutical composition is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., oligonucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods of the disclosure further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., oligonucleotide) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

In some embodiments, compositions of the disclosure are administered in a delivery vehicle comprising a nanocarrier selected from the group consisting of a lipid, a polymer and a lipo-polymeric hybrid. In still further embodiments, the first and second polynucleotides are encapsulated in a lipid nanoparticle, polymer nanoparticle, virus-like particle, nanowire, exosome, or hybrid lipid/polymer nanoparticle. In some embodiments, the first and second polynucleotides are encapsulated in the same nanocarrier. In still further embodiments, the first and second polynucleotides are encapsulated in different nanocarriers. In some embodiments, the lipid nanoparticle is ionizable.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

In some embodiments, the composition is administered to the subject as a dose of about 5 mg/kg to about 10 mg/kg. In some embodiments, the composition is administered to the subject as a dose of about 5 mg/kg. In some embodiments, the composition is administered to the subject as a dose of about 10 mg/kg. In some embodiments, the composition is administered to the subject as a dose of about 5.3 mg/kg. In some embodiments, the composition is administered to the subject as a dose of about 10.6 mg/kg.

In some embodiments, the composition is administered to the subject once a week for twelve consecutive weeks. In some embodiments, the composition is administered to the subject once a day for four consecutive days, then once a week for twelve consecutive weeks. In some embodiments, the composition is administered to the subject once a month for at least two consecutive months. In some embodiments, the composition is administered to the subject once every two to three months for at least two consecutive cycles.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion). In some embodiments, the administration of the bottlebrush polymer-oligonucleotide conjugate may be repeated after one day, two days, three days, four days, five days, six days, one week, two weeks, three weeks, four weeks, five weeks, six weeks, seven weeks, two months, three months, four months, five months, six months or longer. The repeated administration may be at the same dose or at a different dose.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the disclosure provides for compositions and methods of modulating or altering the expression of a gene product encoded by a target polynucleotide (e.g., CUG^{exp} mRNA). In some embodiments, the target polynucleotide is a polynucleotide specific to a mammalian cell (e.g., a mammalian cancer cell, a mammalian non-cancer cell, a mammalian muscle cell). In some embodiments, administration to the subject occurs in the absence of a transfection agent. As used herein, “transfection agent” refers to a means of crossing a cell membrane and/or nuclear envelope, wherein the means is not the bottlebrush polymer-oligonucleotide conjugate (i.e., the bottlebrush polymer-oligonucleotide conjugate does not comprise the transfection agent). In some embodiments a transfection agent is a transfection vector or a transfection reagent. In some embodiments, efficacy of administration is determined by measuring the subject's plasma pharmacokinetics, blood availability, extrahepatic distribution, tissue retention, dosing frequency or amount, or a combination thereof.

In another aspect, the disclosure provides a method of producing a bottlebrush polymer-oligonucleotide conjugate, comprising: synthesizing a bottlebrush polymer; and conjugating the bottlebrush polymer with an oligonucleotide comprising a sequence complementary to a region of a pathogenic transcript associated with a muscular dystrophy, thereby producing the bottlebrush polymer-oligonucleotide conjugate.

In some embodiments, synthesizing the bottlebrush polymer comprises polymerizing norbornenyl bromide and norbornenyl PEG; and conjugating the bottlebrush polymer with the oligonucleotide comprises: conjugating the bottlebrush polymer with an azide group, thereby producing an azide-functionalized bottlebrush polymer; and reacting the azide-functionalized bottlebrush polymer and the oligonucleotide, wherein the oligonucleotide is a DBCO-modified oligonucleotide, thereby producing the bottlebrush polymer-oligonucleotide conjugate.

In another aspect, the disclosure provides a method of producing a bottlebrush polymer-oligonucleotide conjugate that comprises synthesizing a bottlebrush polymer; and conjugating the bottlebrush polymer with an ASO, thereby producing the bottlebrush polymer-oligonucleotide conjugate. In some embodiments, synthesizing the bottlebrush polymer comprises polymerizing norbornenyl bromide and norbornenyl PEG. In some embodiments, conjugating the bottlebrush polymer with an ASO comprises conjugating the bottlebrush polymer with an azide group, thereby producing an azide-functionalized bottlebrush polymer; and reacting the azide-functionalized bottlebrush polymer and the ASO, wherein the ASO is a DBCO-modified ASO.

### EXEMPLIFICATION

### Example 1

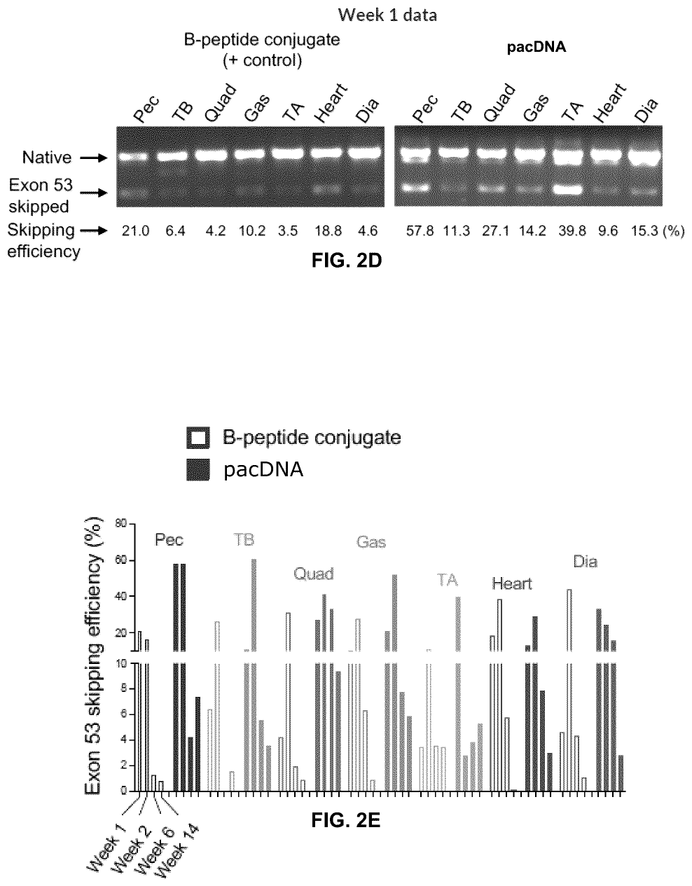

Symptoms of Duchenne muscular dystrophy (DMD) can include loss of voice, cardiomyopathy, respiratory failure, and loss of ambulation, among other possible symptoms. DMD and other dystrophinopathies (e.g., Becker muscular dystrophy) are associated with mutations in the dystrophin gene (DMD) that result in a loss of integrity of the myofiber membrane. Bottlebrush polymer-oligonucleotide conjugates (i.e., BRUSHIELD®DNA, polymer-assisted compaction of DNA (pacDNA)) can improve DMD exon skipping therapeutics. Bottlebrush polymer-oligonucleotide conjugates were tested in Duchenne muscular dystrophy cells (FIG. 2B) and at 1 week post-treatment in mouse models (FIG. 2C; future data to include 2, 4, and 8 weeks post-treatment). The data suggest high potency compared with the state of the art.

### DMD mdx Mice Study Protocol

To study the restoration of dystrophin expression in dystrophin exon 53-deficient (FIG. 2A) mdx male mice (mdx 4cv mice), pacDNA and controls (20 nanomoles, ASO basis (i.e., a dose of pacDNA comprising 20 nanomoles of ASO)) were injected intravenously (i.v.) into 5-6 week old mice (n=4, one dose). The animals were separated into experimental groups: untreated (PBS control), B peptide-conjugated ASO (FIGS. 2D, 2E), L26-pacDNA (L26=ASO1), L31-pacDNA (L31=ASO2), and scrambled pacDNA. Wild-type mice C57BL/6 were used as control for normal dystrophin levels. Animals were sacrificed 1 week post-injection. Muscle samples (pectorals (Pec), quadriceps (quad), tibialis anterior (TA), gastrocnemius (Gas), triceps brachii (TB), diaphragm (dia), and heart muscles (heart)) were harvested, dissected immediately, snap-frozen in liquid nitrogen, and stored at −80° C. for RT-PCR and Western blotting analysis.

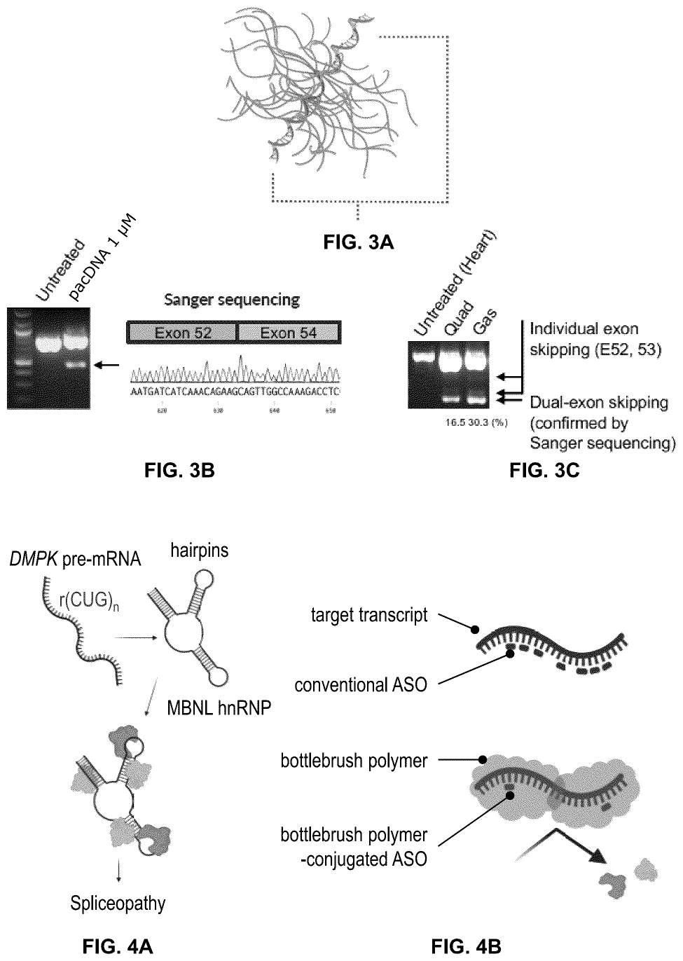

Separately, an exon 52/53 dual-skipping conjugate was tested in mdx4cv mice (FIGS. 3A, 3C) and in myotubes differentiated from C2C12 cells (FIG. 3B). Additional studies contemplated include exon 23-skipping in an mdx mouse model and splicing switching in an EGFP-645 mouse model.

### RT-PCR Analysis

Total RNA from the muscles of wild-type (WT) and mdx mice were extracted. RT-PCR was used to detect dystrophin mRNA using the SuperScript III One-Step RT-PCR system (Thermo Fisher Scientific, Waltham, MA, USA). The calculation of the exon 53 skipping efficiency is based on the following formula: exon 53-skipped transcript intensity/(native+intermediate+exon 53-skipped transcript intensities)×100(%).

### Serum Creatine Kinase Levels

Blood from each group was collected weekly post-injection. The biochemical markers creatine kinase (CK) was assayed to monitor potential broad correction of dystrophin and protection of the sarcolemma throughout the body. The study was continued until the CK level increased for two consecutive weeks (data not shown).

### Western Blot

Protein from muscle tissues of WT and mdx 4cv mice were collected. Thirty micrograms from tissues were used for sodium dodecyl sulfate-polyacrylamide gel electrophoresis (SDS-PAGE). The membranes were incubated with a rabbit polyclonal antibody against dystrophin C-terminal (ab15277; Abcam, Waltham, MA, USA), followed by secondary horseradish peroxidase (HRP)-conjugated anti-rabbit IgG antibody. Signals were detected with the ECL select detection reagent (Cytiva, Global Life Sciences Solutions USA LLC, Marlborough, MA). Vinculin was detected on the same membrane as a loading control. Data not shown.

### Immunohistochemistry and Histology

For immunofluorescence analysis, cryostat-cut sections of quadriceps, tibialis anterior, the gastrocnemius, the triceps brachii, the diaphragm, and the heart muscles were stained with anti-dystrophin antibody (ab15277 antibody, Abcam). Alexa 568 (Abcam) was used as a secondary antibody. Hematoxylin and eosin (H&E) staining was also be performed. Data not shown.

### Muscle Distribution and FACS Sorting

Male mdx 4cv mice (n=3) were treated i.v. with Cy5-labeled PACL31 (20 nmol, ASO basis). All muscle samples were harvested 24 hours post-injection. Samples were immediately frozen in optimum cutting temperature (O.C.T.) compound, sectioned, stained with Hoechst 33342 nuclear counterstain, and imaged using a confocal laser scanning microscope. Data not shown.

FACS analysis was performed to examine uptake among populations of mononucleated cells typically found in skeletal muscle, including (and identified by the associated cell surface marker) muscle satellite cells (MuSCs) (VCAM1+), endothelial cells (CD31+), hematopoietic cells (CD45+) and mesenchymal stem cells (Sca1+). Data not shown.

### Example 2

### Materials and Reagents

Methoxy polyethylene glycol (PEG) Amine, HCl Salt (Catalog No. A3052) was purchased from JenKem Technology USA (Plano, TX). Phosphoramidites and supplies for DNA synthesis were obtained from Glen Research Co., USA (Sterling, VA). All other materials were obtained from MilliporeSigma, Fisher Scientific Inc., USA (Waltham, MA), or VWR International LLC., USA (Radnor, PA), and were used as received unless otherwise indicated.

### Instrumentation

Reversed-phase high-performance liquid chromatography (RP-HPLC) was performed on a Waters (Waters Co., Milford, MA, USA) Breeze 2 HPLC system coupled to a SYMMETRY© C18 3.5 m, 4.6×75 mm reversed-phase column and a 2998 photodiode array (PDA) detector, using triethylammonium acetate (TEAA) buffer (0.1 M) and HPLC-grade acetonitrile as mobile phases. Aqueous gel permeation chromatography (GPC) measurements were performed on a Waters Breeze 2 GPC system equipped with Ultrahydrogel™ 1000, Ultrahydrogel™ 500 and Ultrahydrogel™ 250, 7.8 Å×300 mm column and a 2998 PDA detector (Waters Co., MA, USA). Phosphate-buffered saline (PBS, pH 7.4) was used as the eluent running at a flow rate of 0.8 mL/min.

N,N-dimethylformamide (DMF) GPC was performed on a TOSOH EcoSEC HLC-8320 GPC system (Tokyo, Japan) equipped with a TSKGel GMIIHR-H 7.8×300 mm column and refractive index (RI)/ultraviolet (UV)-visible (Vis) detectors. HPLC-grade DMF with 0.05 M LiBr was used as the mobile phase, with samples run at a 0.5 mL/min flow rate. GPC calibration was performed with polyethylene glycol/polyethylene oxide READYCAL® set (Sigma Aldrich, St. Louis, MO, USA.).

### Cell Culture and Animals

Human DM1 patient-derived fibroblasts with an approximately 2000 CTG repeat mutation in the DMPK gene (cell line GM03989) and healthy control fibroblasts (cell line GM07492) were obtained from Coriell Cell Repositories (Coriell Institute for Medical Research, Camden, NJ, USA). Cells were passaged and cultured in Eagle's Minimum Essential Medium (EMEM) with 10% fetal bovine serum (FBS), 1% antibiotic antimycotic, and 1% non-essential amino acids solution in 5% CO₂ at 37° C. All cell counting was performed using Trypan Blue and a hematocytometer instrument. Cells were frozen for storage in 90% FBS with 10% dimethyl sulfoxide (DMSO, 1 mL).

Homozygous HSA-LR20b transgenic mice (human skeletal actin long repeat line 20b, Strain #032031; abbreviated as HSA-LR or HSA^{LR}) and FVB/N (Friend Virus B NIH Jackson, Strain #001800) were obtained from The Jackson Laboratory, Bar Harbor, ME, USA.⁶ CD-1® mice were obtained from Charles River Laboratories (Wilmington, MA, USA). Animal experiments were conducted at Northeastern University and carried out in accordance with approved Institutional Animal Care and Use Committee guidelines (protocol number: 22-0309). Adult mice aged about 8 weeks were injected via tail vein with pacDNA, cell-penetrating B-peptide (SEQ ID NO: 2) conjugated DNA,⁵⁸ free DNA, or scramble/brush polymer controls dissolved in 200 μL of sterile PBS. Animals were humanely sacrificed by CO₂ at indicated time points for tissue collection. Blood for serum analysis assays was collected from the submandibular vein at indicated time points.

### Antisense Oligonucleotides

Custom DNA oligonucleotides were synthesized on a DR. OLIGO® 48 DNA synthesizer (Biolytic Lab Performance, Inc., Fremont, CA, USA). Short-length single-stranded DNA with a sequence of 5′-CAGCAGCAG-3′ of locked nucleic acid (LNA) modified bases was made, and dT-DBCO (DBCO-dT-CE phosphoramidite: 5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite)) was attached to the 5′ end for downstream “click” chemistry reactions. All-LNA scramble control oligonucleotides (5′-CGCACAAGG-3′) were synthesized in parallel. A fluorescent probe with a sequence of 5′-(Cy3)-CAGCAGCAGCAGCAGCAGCA-3′ (Bold/underline=LNA modification; Cy=cyanine fluorescent dye) (SEQ ID NO: 1) was synthesized for fluorescence in situ hybridization (FISH) assays.⁷¹ All DNA was synthesized using 1 micromole controlled pore glass with support-bound T nucleoside (CPG(T)) columns, or 200 nanomole Cy3/Cy5 CPG columns. Oligonucleotides were cleaved from the CPG solid support and deprotected in aqueous ammonium hydroxide solution (28-30% NH₃ basis) at room temperature for 16 hours. Free oligonucleotides were purified by RP-HPLC. If necessary, dimethoxytrityl (DMT) protecting groups were removed by treatment with 20% acetic acid for 1 hour followed by three extractions in ethyl acetate. Purified DNA was quantified on a NANODROP® ND-1000 UV-Vis Spectrophotometer and lyophilized for storage at −20° C.

Synthesis and Purification of pacDNA Conjugates

Norbornenyl bromide was synthesized using a two-step synthesis involving maleimide (1 equivalent (equiv.)), furan (1 equiv.), 1,4-dibromobutane (4 equiv.), and K₂CO₃ (5 equiv.) as previously described.³⁵ 5-norbornene-2-acetic acid succinimidyl ester (norbornenyl NHS ester) was synthesized via a reaction of exo-5-norbornene carboxylic acid (MilliporeSigma, 1 equiv.) and N-hydroxysuccinimide (Thermo Fisher Scientific (Waltham, MA, USA), 1.4 equiv.) in the presence of 1-Ethyl-3-(3-dimethylaminopropyl)carbodiimide (EDCI) (MilliporeSigma, 1.3 equiv.) as a carboxyl activating agent in dichloromethane (DCM) for 16 hours at room temperature. The product was purified by silica column chromatography with a 2:1 hexane:ethyl acetate mobile phase. Norbornene-PEG (i.e., norbornenyl PEG) was synthesized as previously described by reacting norbornenyl NHS ester (1.5 equiv.) and methoxy-PEG Amine HCl salt (JenKem Technology USA, 1 equiv.) in the presence of N,N-diisopropylethylamine (DIPEA) (Thermo Fisher, 1.5 equiv.) in DCM for 16 hours at room temperature.⁷² The product was precipitated in cold anhydrous ethyl ether, washed three times, and lyophilized for storage at −20° C. Third-generation Grubbs' Catalyst was made by reacting 2^{nd} Generation Grubbs' Catalyst (MilliporeSigma, 1 equiv.) with 3-bromopyridine (MilliporeSigma, 100 equiv.), followed by precipitation in cold hexane as previously described.⁷³

Diblock (i.e., copolymer comprising two different monomers) bottlebrush polymer was synthesized via ring-opening polymerization of norbornenyl bromide and norbornenyl PEG using a 3^{rd} generation Grubbs' catalyst.³⁵ Purified bottlebrush polymer was conjugated with an azide group by substitution reaction with sodium azide (Thermo Fisher Scientific, 100 equiv.) Azide-functionalized bottlebrush polymer (1 equiv.) and DBCO-modified DNA (2.2 equiv.) were reacted in a standard “click chemistry” reaction for 24 hours at 55° C. as previously described.⁴¹ The conjugated pacDNA product was purified via aqueous gel permeation chromatography (GPC) with a 1 M sodium nitrate mobile phase in an Ultrahydrogel 250 angstrom (Å) column. DMF-GPC was performed to quantify the molecular weight, polydispersity index (PDI), and yield of the bottlebrush polymer. After DNA conjugation and purification, the pacDNA was desalted using a NAP®-25 column (Cytiva, Global Life Sciences Solutions USA LLC, Marlborough, MA) and lyophilized for storage at −20° C.

### Fluorescence In Situ Hybridization

Human fibroblasts were seeded in a 24-well confocal plate and treated with various concentrations of pacDNA or controls for 24-72 hours. Then, cells were fixed in 4% paraformaldehyde (PFA) at 4° C. followed by permeabilization with 70% ethanol overnight. Pre-hybridization was then performed using a buffer (30% formamide, 2× saline-sodium citrate (SSC; 2×=0.30 M NaCl and 0.03 M sodium citrate) buffer) for 10 minutes. Hybridization was performed using a FISH probe 5′-(Cy3)-CAGCAGCAGCAGCAGCAGCA-3′ (Bold/underline=LNA modified, 2 ng/μL) (SEQ ID NO: 1) in a buffer containing formamide, SSC, bovine serum albumin (BSA), yeast transfer RNA (tRNA), dextran sulfate, and vanadyl ribonucleoside complex.⁷¹ Cells were stained with DAPI (4′,6-diamidino-2-phenylindole, Thermo Fisher Scientific) for 10 minutes and washed three times with PBS. Images were acquired on a ZEISS® LSM 880 confocal laser scanning microscope (Carl Zeiss Ltd., Cambridge, UK). Foci were counted by eye in about 160 nuclei in each sample group.

### RNA Isolation

HSA-LR20b or FVB/N mice were euthanized, and muscle samples were collected from seven sections (pectoral, triceps brachii, quadriceps, gastrocnemius, tibialis anterior, heart, and diaphragm), flash frozen in liquid nitrogen and stored at −80° C. Individual sections were homogenized in lysis buffer containing 10% β-mercaptoethanol (BME) on a Beadblaster 24R Microtube Homogenizer (Benchmark Scientific, Sayreville, NJ, USA). Total cellular RNA was extracted using the RNEASY® Fibrous Tissue Mini Kit (Qiagen, Germantown, MD, USA) and RNA quality and quantity was assessed on a NANODROP® ND-1000 UV-Vis Spectrophotometer.

### ⁸⁹Zr Radiolabeling to Measure Biodistribution

For radiolabeling, pacDNA was synthesized with two desferrioxamine (DFO, siderophore-derived chelator) functionalities per molecule, which were used to chelate ⁸⁹Zr-oxalate at a ratio of approximately 500 kilobecquerel (kBq) to 1 g. The radiochemical labeling yields were monitored by instant radio-thin-layer chromatography. Radio-fast protein liquid chromatography (FPLC) with a size-exclusion column was used to remove unchelated ⁸⁹Zr. To evaluate biodistribution/clearance and perform small animal positron emission tomography (PET) imaging, C57BL/6 mice were intravenously (i.v.) injected with approximately 500 megabecquerel (MBq) of pacDNA in 100 μL saline. Mice were anesthetized with inhaled isoflurane and re-anesthetized before euthanasia by cervical dislocation at predetermined time points (24 hours, 3 days, 7 days, and 14 days post-injection, n=4 per time point; group size based on prior studies). Organs/tissues of interest were collected, weighed, and radio-counted. Standards were prepared and measured along with the samples to calculate the percentage of the injected dose per gram of tissue (% ID/gram). Positron-emission tomography (PET) scans were carried out at two of the four timepoints (24 hours and 7 days) prior to the sacrifice of the mice. The microPET images were corrected for attenuation, scatter, normalization, and camera dead time and co-registered with microCT images.

### RNA Sequencing and CUG-repeat RATA Quantification

Isolated RNA from quadriceps was sent for total RNA sequencing (Novogene, Durham, NC) targeting 40 million reads per sample. After polyA enrichment and quality control, libraries were sequenced on an ILLUMINA® PE150 system to obtain paired-end reads of 150 base pairs (bp). After trimming and alignment, reads were mapped to the mouse genome (mm39 assembly), and analysis was performed in RStudio using the DESeq2, clusterProfiler, fgsea, and Rsubread packages. Gene set enrichment analysis (GSEA) was performed to assess functional gene expression changes after treatment. Splicing events were calculated using the rMATS statistical model.⁷⁴ Significant splice events were classified as having an adjusted p value (padj)<0.01. Adjusted p values (padj<0.05) and absolute value of log₂(fold change) (|log₂ fc≡)>1 were used to filter differentially expressed genes (DEGs).

For quantification of mutant CUG-repeat RNA, FASTQ files were read, transformed, and analyzed using the FS2 library in Scala. Regular expressions were used to identify and count all instances of uninterrupted “CAG” trinucleotide repeats within the reads of at least 17 consecutive repeats. This cutoff was selected based on the near complete absence of reads containing at least 17 repeats within 5 wild-type samples (<12 normalized occurrences per 17.55 million reads). The total nucleotides in repeat segments were divided by the total nucleotides in the sample to calculate the fraction of the sample composed of repeat segments. This fraction was then divided by the average fraction of the 2 nontreated DM1 samples for a relative repeat amount calculation. Levels of endogenous genes containing short (2-25) CUG-repeat RNA tracts were examined for potential off-target effects of the pacDNA.

Myotonia and Grip Strength in pacDNA-Treated Mice

A hindlimb pinching experiment was used to assess skeletal muscle myotonia. Mice were lightly pinched about 1 cm above the base of the tail 15 times. The response to each pinch was classified as severe myotonia (>1 second for muscles to relax, score of 3), myotonia (0.5-1 second, 2), quick recovery myotonia (<0.5 seconds, 1) myotonia, single-leg myotonia (0.5), or no myotonia (0). Mice were tested in a rotating order and given a 20-minute break after 8 pinches. Mice were weighed each week on a standard animal scale.

Forelimb/hindlimb grip force was measured on a Grip Strength Meter (Bioseb, Pinellas Park, FL, USA). Measurements were collected at baseline and various time points during and after the treatment period and compared to age and sex-matched nontreated HSA-LR20b and FVB/N wild-type controls. The average of four tests was recorded for each mouse.

### Example 3

### PacDNA Enhanced ASO Muscle Bioavailability and Enabled the Targeting of CUG^{exp} Nuclear Foci

Polyethylene glycol (10 kDa) based brush polymers were synthesized by ring-opening metathesis polymerization (ROMP) (FIG. 5A) and purified by size exclusion chromatography (SEC; FIG. 5B) using methods that were previously described³⁵. Custom oligonucleotides were conjugated to the polymer backbone via a “click” chemistry reaction.⁴¹ The uniformity and morphology of these conjugates were assessed by transmission electron microscopy (TEM). Conjugates were highly uniform and exhibited a spherical morphology (FIG. 5C).

The 14-day biodistribution of intravenous (i.v.) injected pacDNA in CD-1® mice was measured using ⁸⁹Zr-radiolabeled conjugates. A 100-fold increase in muscle pacDNA concentration was observed at 1 day postinjection compared to free DNA (FIGS. 5D, 5E, 5F). This enhanced accumulation, also seen in cardiac muscle, persisted at 14 days postinjection, while levels of free DNA dropped to one-hundredth of their 1 day level at 14 days.

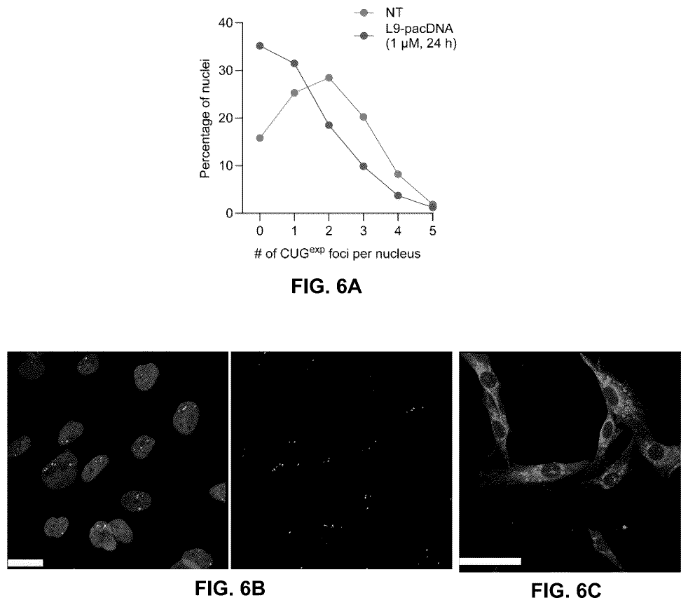

Fluorescence in situ hybridization (FISH) was performed to detect and quantify the presence of nuclear CUG trinucleotide repeat expansion (CUG^{exp}) foci in primary human DM1 fibroblasts with a (CTG)₂₀₀₀ repeat mutation in the DMPK gene (GM03989, Coriell Repositories). Using a fluorescent FISH probe, CUG^{exp} foci were detected in the nuclei of these cells (FIG. 6B). After incubation with 1 μM L9-pacDNA for 24 hours, the number of foci detected per nucleus decreased (FIG. 6A). The percentage of nuclei with 0 foci increased from 15.8% to 35.2%, and the most common number of foci per nucleus dropped from 2 to 0. When these cells were treated with fluorescently labeled 1 μM (Cy5)-L9-pacDNA for 24 hours and imaged, the conjugate was detected in both the endosomes of the cells and to a lesser extent inside the nuclei (FIG. 6C). These results indicate that either the entire L9-pacDNA conjugate, or a cytoplasmically cleaved section of the ASO itself, can enter the nucleus and interact with target CUG^{exp} RNA without the assistance of a transfection agent. Phosphodiester-linked locked nucleic acid (LNA)-modified ASOs are negatively charged and are likely able to passively diffuse through the positively charged nuclear pore complex to reach their targets.⁴²⁻⁴⁵

### Example 4

### L9-pacDNA Partially Corrected DM1-Associated Alternative Splicing in HSA-LR20B Mice

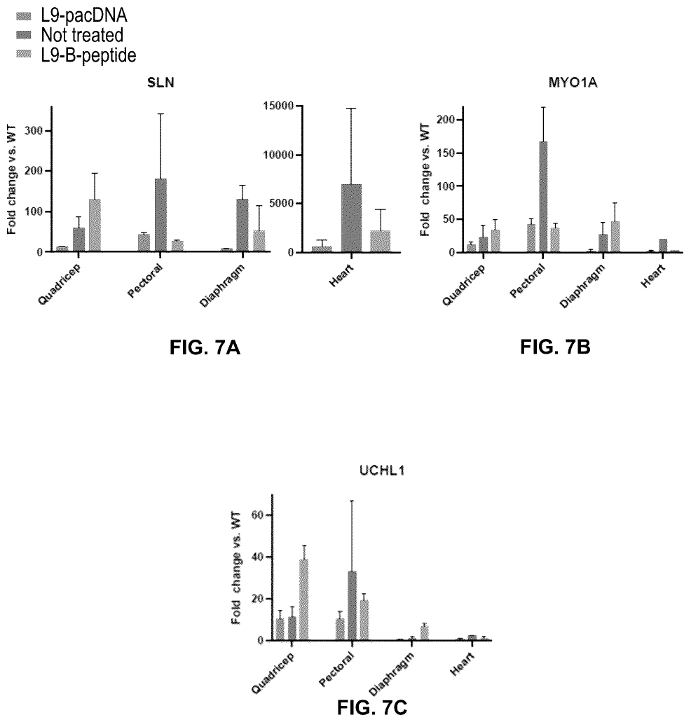

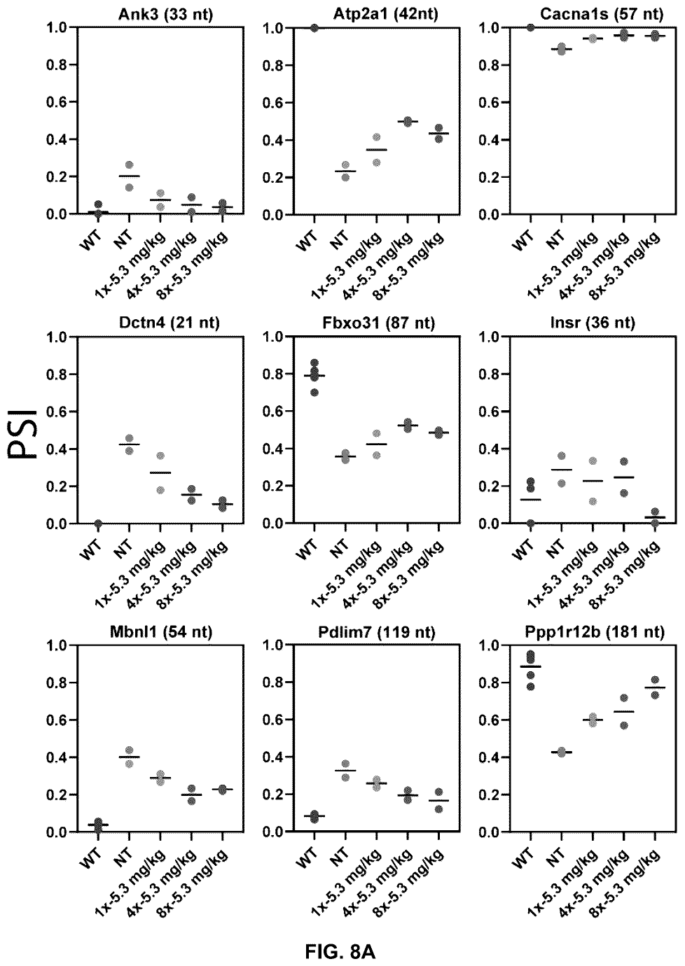

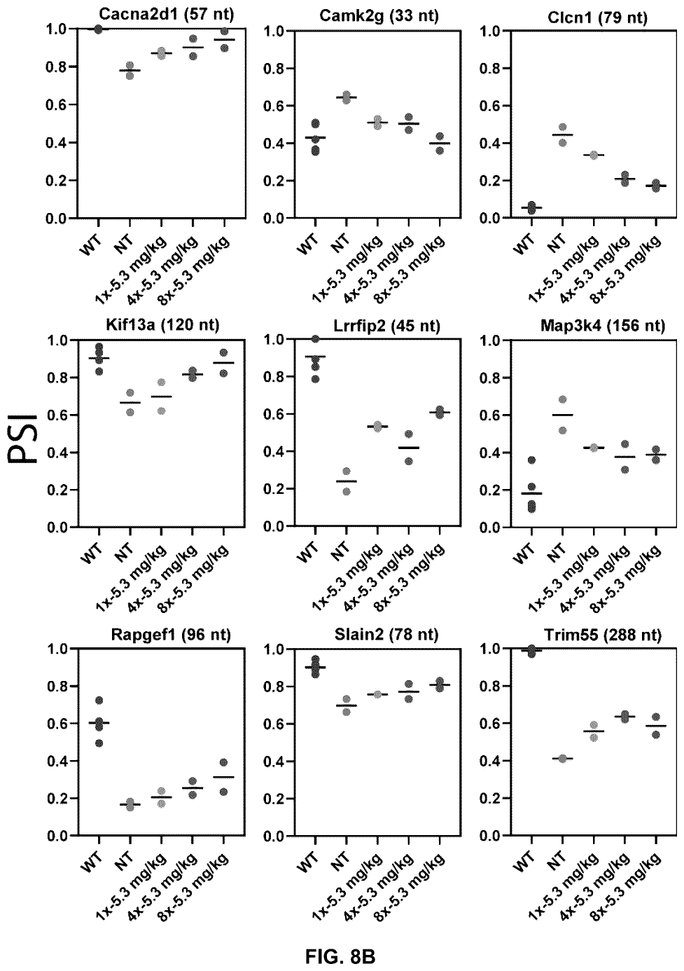

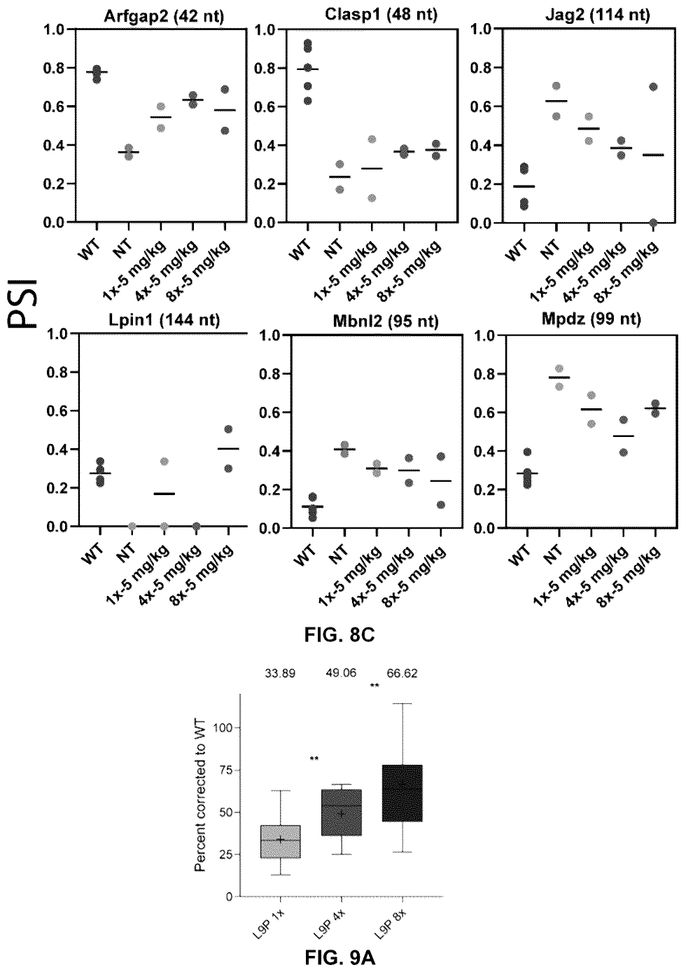

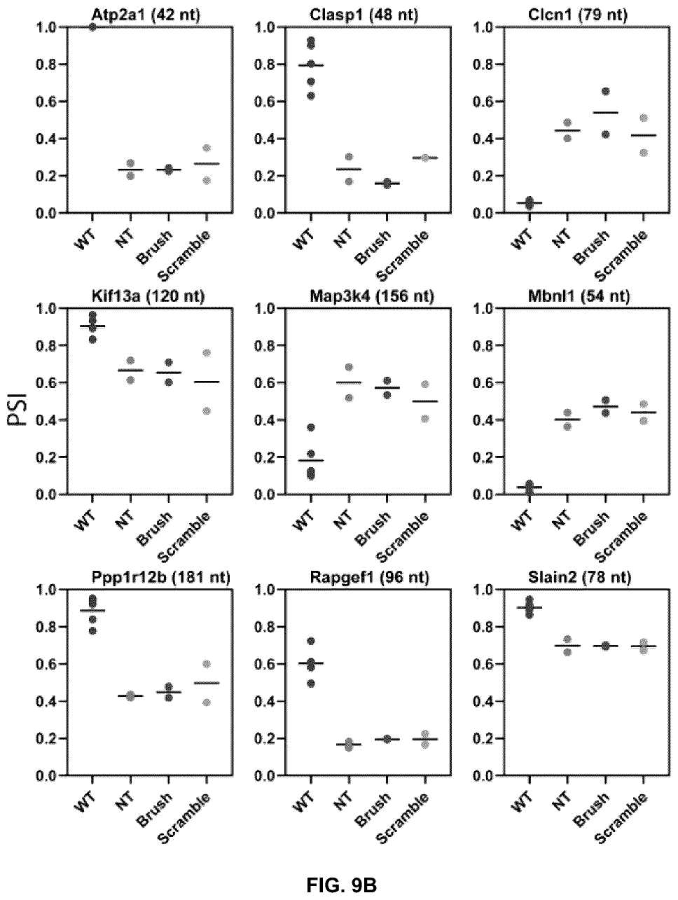

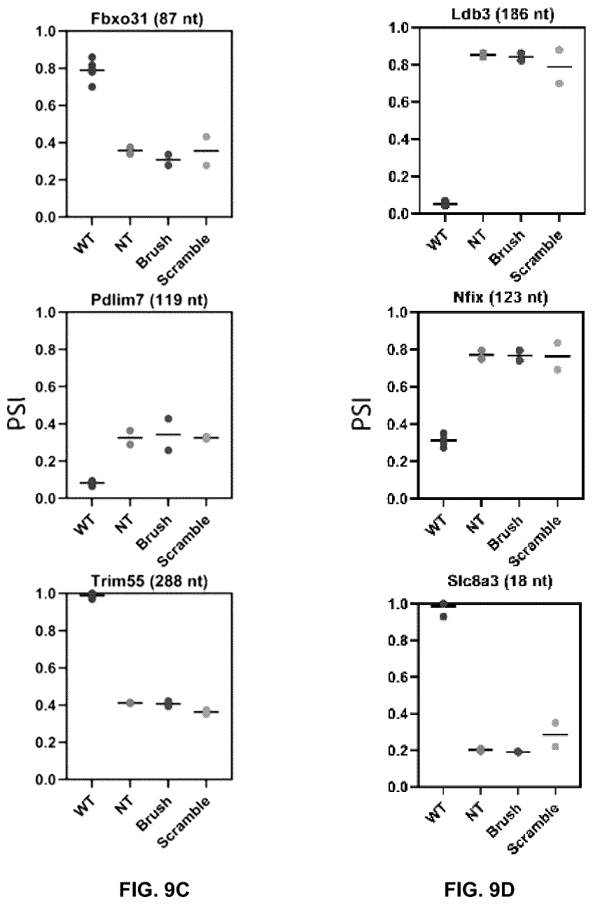

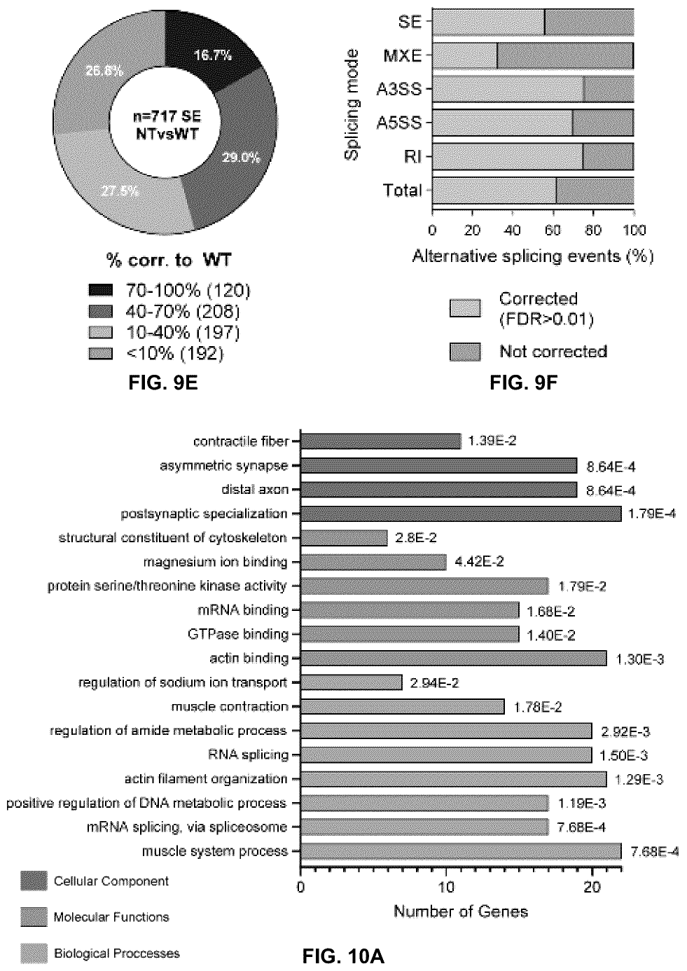

HSA-LR20b mice (about 8 weeks old) were i.v. injected by tail vein with one or multiple doses of 5.3 mg/kg (40 nanomoles, ASO basis (i.e., a dose of bottlebrush polymer-ASO conjugate comprising 40 nanomoles of ASO)) of L9-pacDNA or controls (Table 1). Two weeks after the final injection, mice were sacrificed to assess the therapeutic effect. Total cellular RNA from mouse quadriceps was sequenced to evaluate gene expression, splicing, and relative CUG-repeat RNA levels. Among the many genes affected in DM1 (see, e.g., FIGS. 7A-7C) are several encoding membrane ion channel proteins. Deficiencies in these proteins disrupt normal muscle ion gradients and electrophysiology, leading to hallmark physical manifestations of DM1 such as myotonia, myopathy, and arrhythmia.⁴⁶⁻⁵⁰ Exon inclusion, also known as percent spliced-in (PSI), was measured across a panel of 18 exonic gene fragments reported to experience mis-splicing in DM1-affected muscles (FIGS. 8A, 8B, 8C).⁵¹⁻⁵⁶ Splicing dysregulation improved two weeks after pacDNA treatment, and most splicing events seemed to respond to treatment in a dose-dependent manner. Across the entire panel, the 1-dose, 4-dose, and 8-dose cohorts exhibited average splicing corrections of 33.89%, 49.06%, and 66.62%, respectively (FIG. 9A). Notably, several transcripts associated with specific DM1 phenotypes (e.g., CLCN1, INSR, CACNA1S)^{48, 57} displayed strong splicing correction nearly back to wild-type levels. In mice injected with 40 nanomoles of free LNA-modified (CAG)₃ ASO (L9-free), the average percent splicing correction across this 18-gene fragment panel was just 13.46%. A single 40-nanomole injection of a positive control L9-B-peptide (cationic B-peptide⁵⁸ (CAG)₃ ASO conjugate) achieved a 34.06% average splicing correction in this panel. One 40-nanomole injection of free brush polymer (no conjugated ASO) or pacDNA-scramble ASO (L9^{scr}-pacDNA) negative controls failed to correct alternative splicing in DM1-related genes (FIGS. 9B, 9C, 9D).

TABLE 1
Oligonucleotide and peptide sequences.

| | | | SEQ |
|---|---|---|---|
| | | | ID |
| Name | Description | Sequence | NO. |
| L9-free | L9 ASO, not conjugated | 5′-CAGCAGCAG-3′ | |
| | to bottlebrush polymer; | | |
| | MW = 3027 g/mol | | |
| L9-pacDNA | bottlebrush polymer-L9 | Brush-5′-CAGCAGCAG-3′ | |
| | conjugate; MW = | | |
| | approximately 303,027 | | |
| | g/mol | | |
| L9^{scr}-pacDNA | bottlebrush polymer- | Brush-5′-CGCACAAGG-3′ | |
| | scrambled L9 conjugate | | |
| FISH Probe | for detection of (CUG)ₙ | 5′-(Cy3)-CAGCAGCAGCA | 1 |
| | RNA | GCAGCAGCA-3′ | |
| B-peptide | cell-penetrating peptide | RXRRBRRXRRBRXB | 2 |
| L9-B- | B-peptide-L9 conjugate | B-peptide-5′- | |
| peptide | | CGCACAAGG-3′ | |

Bold/underline = locked nucleic acid (LNA) modified DNA bases, DBCO (dibenzocyclooctyne), Cy-3 (Cyanine 3), R (L-arginine), X (6-aminohexanoic acid), B (β alanine).

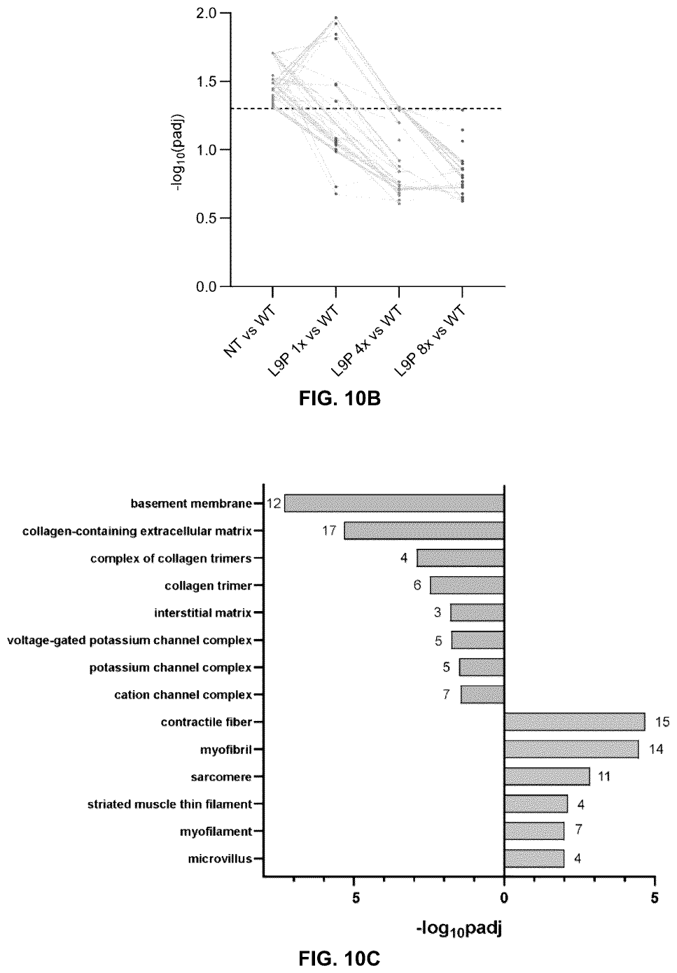

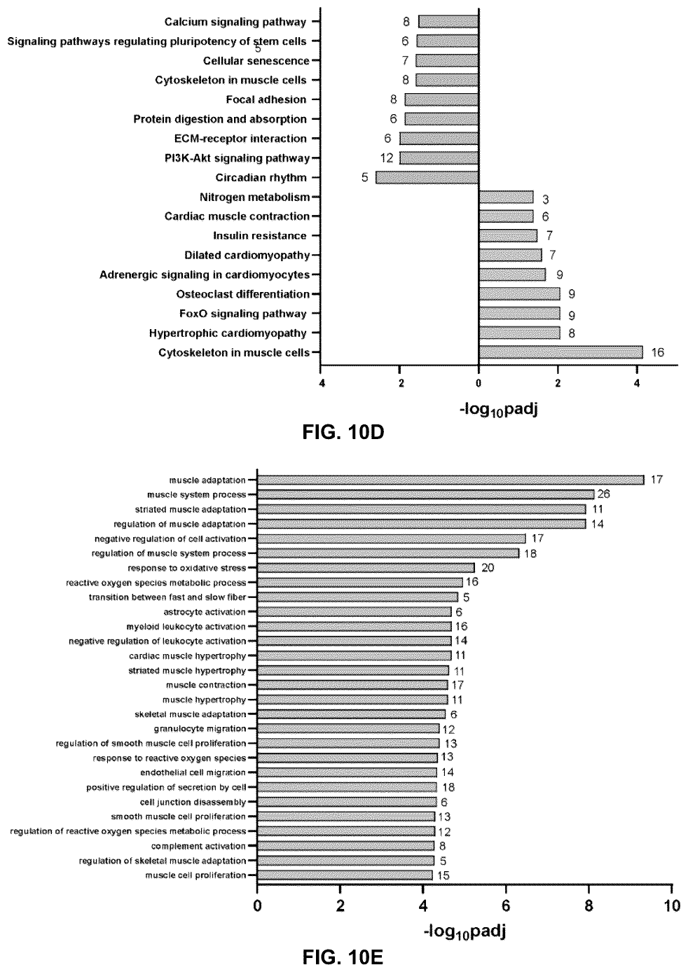

Outside this panel, L9-pacDNA treatment also corrected a significant fraction of the global alternative splicing. Of 717 skipped exon (SE) events significant in nontreated HSA-LR20b mice vs. wild-type and detected after four doses (4×) of L9-pacDNA, treatment corrected the PSI of 120 events by 70-100%, 208 events by 40-70%, and 197 events by 10-40% (FIG. 9E). Without being bound by theory, percent splicing correction across various splicing modes following 4×L9-pacDNA treatment suggested that L9-pacDNA treatment, likely through the release of trapped MBNL1 proteins, led to correction of a large subset of splicing abnormalities in each splicing mode (e.g., alternative 3′ end splice sites (A3SS), alternative 5′ end splice sites (A5SS), mutually exclusive exons (MXE) splicing events, retained-introns (RI), and skipped-exons (SE)) (FIG. 9F). 402 SE events corrected (FDR>0.01 vs WT) by 4×L9-pacDNA were analyzed by gene set enrichment analysis (GSEA) revealing functional signatures associated with the corrected splicing (FIG. 10A). These included muscle contraction, ion channel activity, RNA splicing, metabolic function, and other categories known to be impaired in DM1. Additional GSEA is shown in FIGS. 10B-10E and Table 2 (downregulated genes defined as log₂ fc<−1 and padj<0.05; columns display padj of these same biological processes (BP) in the downregulated genes of 1×, 4×, and 8×pacDNA (L9-P) treated mice relative to wild type).

TABLE 2
Biological processes (BP) associated with downregulated genes
in HSA-LR20b mice relative to wild-type.

| | No | | | |
|---|---|---|---|---|
| | Treatment vs | 1× L9-P vs | 4× L9-P vs | 8× L9-P vs |
| Enriched BP | WT | WT | WT | WT |
| negative | 1.708 | ND | 0.740 | 0.679 |
| regulation of | | | | |
| protein secretion | | | | |
| (GO: 0050709) | | | | |
| negative | 1.703 | 1.082 | 0.737 | ND |
| regulation of | | | | |
| lipid metabolic | | | | |
| process | | | | |
| (GO: 0045833) | | | | |
| negative | 1.703 | ND | 0.879 | 0.624 |
| regulation of | | | | |
| insulin secretion | | | | |
| (GO: 0046676) | | | | |
| negative | 1.703 | 1.845 | 1.197 | 0.639 |
| regulation of | | | | |
| steroid | | | | |
| biosynthetic | | | | |
| process | | | | |
| (GO: 0010894) | | | | |
| negative | 1.703 | ND | 1.310 | ND |
| regulation of | | | | |
| peptide hormone | | | | |
| secretion | | | | |
| (GO: 0090278) | | | | |
| negative | 1.703 | 1.811 | 1.197 | ND |
| regulation of | | | | |
| steroid | | | | |
| metabolic | | | | |
| process | | | | |
| (GO: 0045939) | | | | |
| negative | 1.703 | ND | 1.310 | ND |
| regulation of | | | | |
| peptide secretion | | | | |
| (GO:0002792) | | | | |
| positive | 1.703 | 1.967 | 1.310 | 1.146 |
| regulation of | | | | |
| osteoblast | | | | |
| differentiation | | | | |
| (GO:0045669) | | | | |
| lipid catabolic | 1.703 | 0.729 | 0.841 | ND |
| process | | | | |
| (GO:0016042) | | | | |
| negative | 1.703 | ND | 0.686 | ND |
| regulation of | | | | |
| protein transport | | | | |
| (GO: 0051224) | | | | |
| small molecule | 1.703 | ND | 0.667 | ND |
| catabolic process | | | | |
| (GO: 0044282) | | | | |
| negative | 1.703 | ND | 0.678 | ND |
| regulation of | | | | |
| establishment of | | | | |
| protein | | | | |
| localization | | | | |
| (GO: 1904950) | | | | |
| regulation of | 1.544 | 0.678 | 0.632 | 0.652 |
| lipid metabolic | | | | |
| process | | | | |
| (GO: 0019216) | | | | |
| negative | 1.544 | ND | 1.197 | ND |
| regulation of | | | | |
| hormone | | | | |
| secretion | | | | |
| (GO: 0046888) | | | | |
| Notch signaling | 1.514 | 1.473 | 0.841 | 0.862 |
| pathway | | | | |
| (GO: 0007219) | | | | |
| negative | 1.514 | ND | 0.841 | ND |
| regulation of | | | | |
| secretion by cell | | | | |
| (GO: 1903531) | | | | |
| regulation of | 1.488 | 1.967 | 1.310 | 0.918 |
| glucocorticoid | | | | |
| metabolic | | | | |
| process | | | | |
| (GO:0031943) | | | | |
| aldosterone | 1.488 | 1.967 | 1.310 | 0.918 |
| biosynthetic | | | | |
| process | | | | |
| (GO: 0032342) | | | | |
| negative | 1.488 | 1.967 | 1.310 | 0.918 |
| regulation of | | | | |
| hormone | | | | |
| biosynthetic | | | | |
| process | | | | |
| (GO: 0032353) | | | | |
| negative | 1.488 | ND | 0.606 | ND |
| regulation of | | | | |
| neuron apoptotic | | | | |
| process | | | | |
| (GO: 0043524) | | | | |
| negative | 1.488 | ND | 0.766 | ND |
| regulation of | | | | |
| secretion | | | | |
| (GO: 0051048) | | | | |
| insulin-like | 1.488 | 1.481 | 0.922 | 1.064 |
| growth factor | | | | |
| receptor | | | | |
| signaling | | | | |
| pathway | | | | |
| (GO: 0048009) | | | | |
| mineralocorticoi | 1.488 | 1.967 | 1.310 | 0.898 |
| d biosynthetic | | | | |
| process | | | | |
| (GO: 0006705) | | | | |
| actin-myosin | 1.488 | 1.066 | 0.740 | ND |
| filament sliding | | | | |
| (GO: 0033275) | | | | |
| cortisol | 1.488 | 1.967 | 1.310 | 0.898 |
| metabolic | | | | |
| process | | | | |
| (GO: 0034650) | | | | |
| regulation of | 1.488 | 1.473 | 0.922 | ND |
| alcohol | | | | |
| biosynthetic | | | | |
| process | | | | |
| (GO: 1902930) | | | | |
| regulation of | 1.488 | 1.079 | 1.071 | ND |
| steroid | | | | |
| metabolic | | | | |
| process | | | | |
| (GO: 0019218) | | | | |
| low-density | 1.448 | 1.058 | 0.740 | ND |
| lipoprotein | | | | |
| particle | | | | |
| remodeling | | | | |
| (GO: 0034374) | | | | |
| regulation of | 1.438 | 1.045 | 0.740 | 0.856 |
| gonadotropin | | | | |
| secretion | | | | |
| (GO: 0032276) | | | | |
| aldosterone | 1.438 | 1.967 | 1.310 | 0.856 |
| metabolic | | | | |
| process | | | | |
| (GO: 0032341) | | | | |
| primary alcohol | 1.438 | 1.967 | 1.310 | 0.856 |
| biosynthetic | | | | |
| process | | | | |
| (GO: 0034309) | | | | |
| negative | 1.438 | 1.045 | 0.740 | ND |
| regulation of | | | | |
| alcohol | | | | |
| biosynthetic | | | | |
| process | | | | |
| (GO: 1902931) | | | | |
| regulation of | 1.398 | 1.034 | 0.740 | ND |
| skeletal muscle | | | | |
| adaptation | | | | |
| (GO: 0014733) | | | | |
| mineralocorticoi | 1.379 | 1.967 | 1.310 | 0.812 |
| d metabolic | | | | |
| process | | | | |
| (GO: 0008212) | | | | |
| negative | 1.362 | 1.355 | 0.841 | ND |
| regulation of | | | | |
| lipid | | | | |
| biosynthetic | | | | |
| process | | | | |
| (GO:0051055) | | | | |
| glucocorticoid | 1.354 | 1.967 | 1.310 | 0.796 |
| biosynthetic | | | | |
| process | | | | |
| (GO:0006704) | | | | |
| negative | 1.354 | 1.967 | 1.310 | 0.796 |
| regulation of | | | | |
| hormone | | | | |
| metabolic | | | | |
| process | | | | |
| (GO:0032351) | | | | |
| immature T cell | 1.336 | 1.003 | 0.716 | 0.768 |
| proliferation in | | | | |
| thymus | | | | |
| (GO:0033080) | | | | |
| immature T cell | 1.327 | 0.994 | 0.714 | 0.744 |
| proliferation | | | | |
| (GO:0033079) | | | | |
| glycerol ether | 1.310 | 0.988 | 0.709 | 0.725 |
| metabolic | | | | |
| process | | | | |
| (GO: 0006662) | | | | |
| ether lipid | 1.310 | 0.988 | 0.709 | 0.725 |
| metabolic | | | | |
| process | | | | |
| (GO: 0046485) | | | | |
| regulation of | 1.310 | 1.922 | 1.287 | 1.291 |
| steroid hormone | | | | |
| biosynthetic | | | | |
| process | | | | |
| (GO: 0090030) | | | | |

GO = gene ontology term; ND = not determined

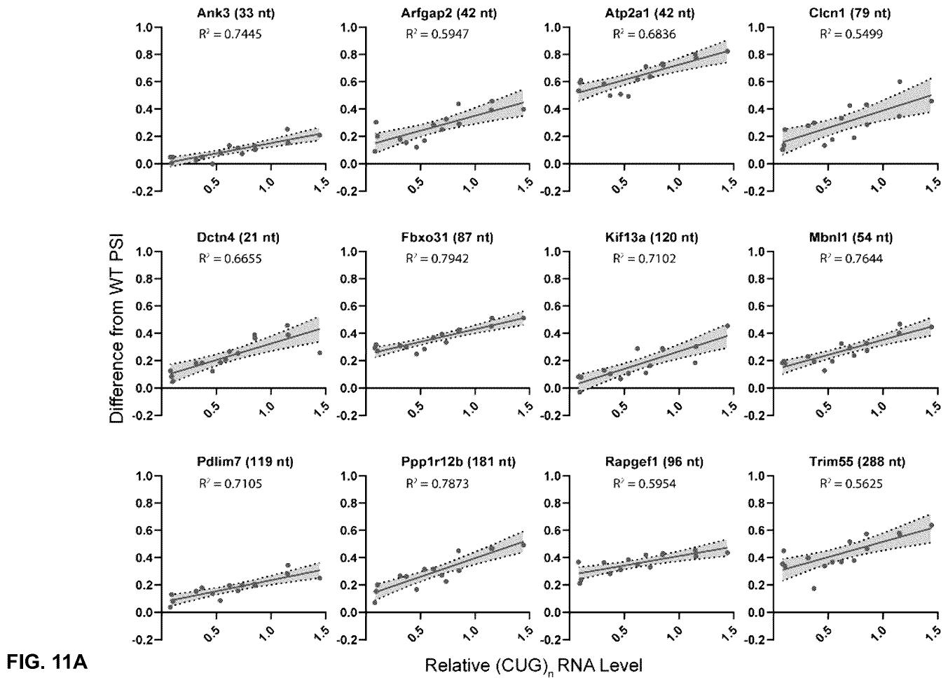

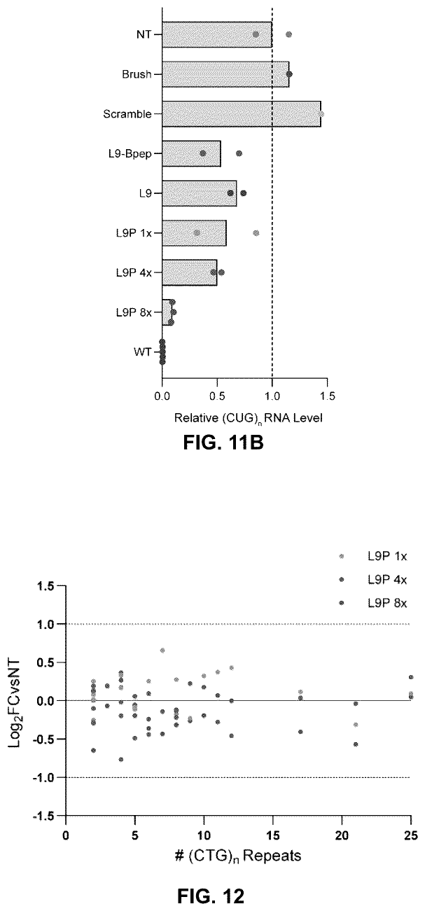

While the exact mechanism is unknown, reduced levels of CUG^{exp} RNA in DM1 may be a prerequisite for splicing correction.^{11, 13} Unaligned RNA-sequencing files can be parsed to identify and quantify the presence of specific motifs, such as the long uninterrupted “CAG”-repeat trinucleotide pattern in cDNA libraries generated from HSA-LR20b mouse RNA.⁵⁹ Doing so allowed for the relative quantification of CUG-repeat RNA in each sample as compared to nontreated controls (FIG. 11B). Plotting the relative level of repeat “CAG” RNA tracts for each sample versus the difference from wild-type splicing inclusion levels in the same sample for 12 DM1-associated splice events revealed a positive relationship between these values (FIG. 11A), with greater levels of repeat RNA correlating with a higher degree of pathogenic splicing.

Since the (CAG)₃ L9 ASO has the potential to bind other endogenous (CUG)ₙ-containing transcripts, the expression levels of 22 genes with varying (2-25) CTG repeats were then examined (Table 3). After 1-dose, 4-dose, or 8-dose treatment with L9-pacDNA, none of these genes were significantly up or downregulated, confirming the absence of off-target short CUG-repeat transcript modulation at these doses (FIG. 12).

TABLE 3
Expression levels of endogenous genes containing short (2-25)
CTG repeats relative to nontreated (NT) HSA-LR20b mice.

| | (CTG)ₙ or | Log₂FC | Log₂FC | Log₂FC |
|---|---|---|---|---|
| Gene | *(CAG)ₙ | L9P 1× vs. NT | L9P 4× vs. NT | L9P 8× Vs. NT |
| Papss2 | 2 | 0.255 | 0.128 | 0.118 |
| Bpgm | 2 | −0.252 | −0.286 | 0.003 |
| Dmpk | 2 | 0.014 | 0.192 | −0.294 |
| Tcf4 | 2 | 0.076 | −0.102 | −0.649 |
| Ltbp3 | 3 | 0.199 | 0.188 | −0.068 |
| Rpl14 | 4 | −0.194 | −0.199 | 0.173 |
| Lrp8 | 4 | 0.165 | −0.767 | 0.364 |
| Bri3bp | 4 | 0.331 | −0.020 | 0.266 |
| Map3k4 | 5 | −0.114 | 0.056 | −0.195 |
| Notch4 | 5 | −0.085 | −0.488 | −0.055 |
| Ptbp1 | 6 | 0.254 | −0.442 | −0.360 |
| Sdc3 | 6 | 0.088 | 0.094 | −0.242 |
| Armcx6 | 7 | 0.656 | −0.140 | −0.433 |
| Mllt3 | 8 | −0.177 | −0.219 | −0.316 |
| Tacc1 | 8 | 0.275 | −0.133 | −0.118 |
| Txlnb | 9 | −0.228 | 0.223 | −0.264 |
| Tnfrsf22 | 10 | 0.324 | 0.175 | −0.195 |
| Pcolce | 12 | 0.431 | −0.458 | −0.002 |
| Fgd4 | 21 | −0.312 | −0.039 | −0.568 |
| Mapkap1 | 25 | 0.093 | 0.305 | 0.050 |
| Nr3cl* | 17 | 0.117 | 0.036 | −0.406 |
| Dap* | 11 | 0.374 | −0.278 | 0.069 |

### Example 5

### L9-pacDNA Alleviated Myotonia and Improved Muscle Strength in HSA-LR20b Mice

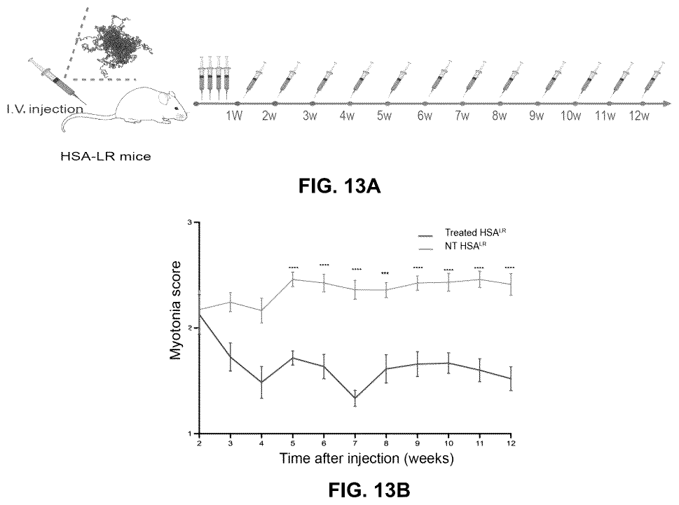

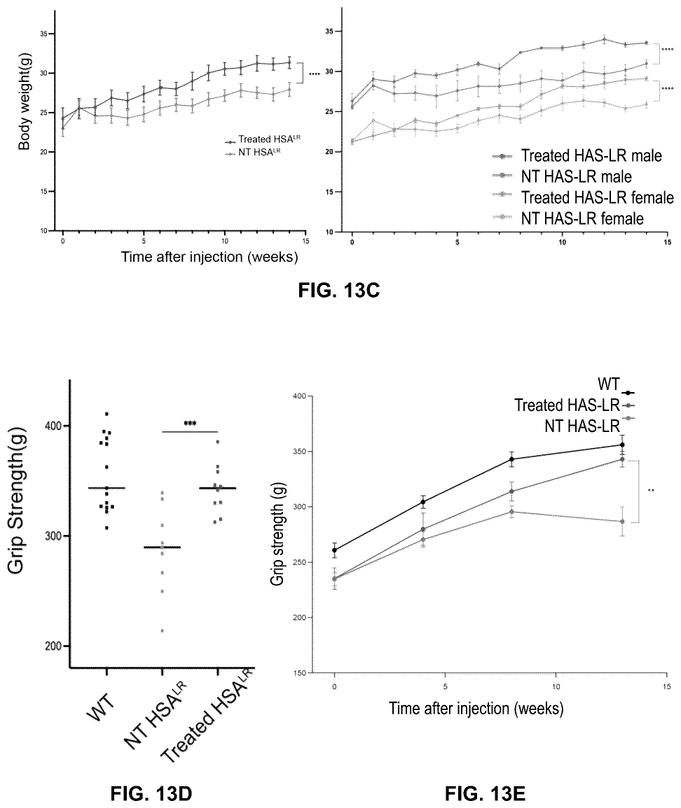

Next, the long-term therapeutic efficacy of the pacDNA was evaluated in HSA-LR20b mice. DM1 in humans is characterized by progressive muscle wasting and weakness, alongside myotonia, which refers to the delayed relaxation of muscles after contraction. Myotonia is a key feature in both DM1 patients and this mouse model, and it contributes significantly to physical impairment. L9-pacDNA was administered continuously to a cohort of mice over 12 weeks. Starting at 8 weeks of age, L9-pacDNA was administered i.v. at 10.6 mg/kg/day for four consecutive days, then weekly (10.6 mg/kg/week) from week 2 to week 12 (FIG. 13A). To evaluate myotonia severity in the mice, a hindlimb pinch test was performed each week, assessing delayed muscle relaxation in vivo. Significant improvements in myotonia recovery time were observed starting in the third week of treatment (p<0.01), with maximal therapeutic effect achieved by the seventh week. Myotonia scores in the treated group remained consistently lower than those in the non-treated group (p<0.001 from weeks 7 to 12) (FIG. 13B). These improvements in myotonia reflected a reduction in a primary symptom of DM1, highlighting the therapeutic potential of the L9-pacDNA. Additionally, mice in the treatment group showed a notable increase in body weight compared to the non-treated group (FIGS. 13C, M5B). To further assess the functional impact of L9-pacDNA treatment, a grip strength test was conducted to evaluate muscle strength in HSA-LR20b mice. At week 12, the treated mice were significantly stronger than their untreated littermates (p<0.001) (FIGS. 13D, 13E). These findings suggested that L9-pacDNA effectively improved muscle strength and alleviated myotonia following long-term treatment.

### REFERENCES

(1) Johnson, N. E.; Butterfield, R. J.; Mayne, K.; Newcomb, T.; Imburgia, C.; Dunn, D.; Duval, B.; Feldkamp, M. L.; Weiss, R. B. Population-Based Prevalence of Myotonic Dystrophy Type 1 Using Genetic Analysis of Statewide Blood Screening Program. Neurology 2021, 96 (7), e1045-e1053. DOI: 10.1212/wnl.0000000000011425.
(2) Overend, G.; Légaré, C.; Mathieu, J.; Bouchard, L.; Gagnon, C.; Monckton, D. G. Allele length of the DMPK CTG repeat is a predictor of progressive myotonic dystrophy type 1 phenotypes. Hum Mol Genet 2019, 28 (13), 2245-2254. DOI: 10.1093/hmg/ddz055.
(3) Kuyumcu-Martinez, N. M.; Wang, G.-S.; Cooper, T. A. Increased steady state levels of CUGBP1 in Myotonic Dystrophy 1 are due to PKC-mediated hyper-phosphorylation. Mol Cell 2007, 28 (1), 68-78. DOI: 10.1016/j.molcel.2007.07.027.
(4) Ho, T. H.; Savkur, R. S.; Poulos, M. G.; Mancini, M. A.; Swanson, M. S.; Cooper, T. A. Colocalization of muscleblind with RNA foci is separable from mis-regulation of alternative splicing in myotonic dystrophy. Journal of Cell Science 2005, 118 (13), 2923-2933. DOI: 10.1242/jcs.02404.
(5) Querido, E.; Gallardo, F.; Beaudoin, M.; Menard, C.; Chartrand, P. Stochastic and reversible aggregation of mRNA with expanded CUG-triplet repeats. Journal of Cell Science 2011, 124 (10), 1703-1714. DOI: 10.1242/jcs.073270.
(6) Mankodi, A.; Logigian, E.; Callahan, L.; McClain, C.; White, R.; Henderson, D.; Krym, M.; Thornton, C. A. Myotonic Dystrophy in Transgenic Mice Expressing an Expanded CUG Repeat. Science 2000, 289 (5485), 1769-1772. DOI: 10.1126/science.289.5485.1769.
(7) Corey, D. R. Nusinersen, an antisense oligonucleotide drug for spinal muscular atrophy. Nature Neuroscience 2017, 20 (4), 497-499. DOI: 10.1038/nn.4508.
(8) Aartsma-Rus, A. The Future of Exon Skipping for Duchenne Muscular Dystrophy. Hum Gene Ther 2023, 34 (9-10), 372-378. DOI: 10.1089/hum.2023.026.
(9) Lin, X.; Miller, J. W.; Mankodi, A.; Kanadia, R. N.; Yuan, Y.; Moxley, R. T.; Swanson, M. S.; Thornton, C. A. Failure of MBNL1-dependent post-natal splicing transitions in myotonic dystrophy. Human Molecular Genetics 2006, 15 (13), 2087-2097. DOI: 10.1093/hmg/dd1132.
(10) Wojtkowiak-Szlachcic, A.; Taylor, K.; Stepniak-Konieczna, E.; Sznajder, L. J.; Mykowska, A.; Sroka, J.; Thornton, C. A.; Sobczak, K. Short antisense-locked nucleic acids (all-LNAs) correct alternative splicing abnormalities in myotonic dystrophy. Nucleic Acids Research 2015, 43 (6), 3318-3331. DOI: 10.1093/nar/gkvl63.
(11) Wheeler, T. M.; Sobczak, K.; Lueck, J. D.; Osborne, R. J.; Lin, X.; Dirksen, R. T.; Thornton, C. A. Reversal of RNA Dominance by Displacement of Protein Sequestered on Triplet Repeat RNA. Science 2009, 325 (5938), 336-339. DOI: 10.1126/science.1173110.
(12) Sobczak, K.; Wheeler, T. M.; Wang, W.; Thornton, C. A. RNA Interference Targeting CUG Repeats in a Mouse Model of Myotonic Dystrophy. Mol Ther 2013, 21 (2), 380-387. DOI: 10.1038/mt.2012.222.
(13) Mulders, S. A. M.; van den Broek, W. J. A. A.; Wheeler, T. M.; Croes, H. J. E.; van Kuik-Romeijn, P.; de Kimpe, S. J.; Furling, D.; Platenburg, G. J.; Gourdon, G.; Thornton, C. A.; et al. Triplet-repeat oligonucleotide-mediated reversal of RNA toxicity in myotonic dystrophy. Proc. Natl. Acad. Sci. U.S.A. 2009, 106 (33), 13915-13920. DOI: 10.1073/pnas.0905780106.
(14) Burki, U.; Keane, J.; Blain, A.; O'Donovan, L.; Gait, M. J.; Laval, S. H.; Straub, V. Development and Application of an Ultrasensitive Hybridization-Based ELISA Method for the Determination of Peptide-Conjugated Phosphorodiamidate Morpholino Oligonucleotides. Nucleic Acid Therapeutics 2015, 25 (5), 275-284. DOI: 10.1089/nat.2014.0528.
(15) González-Barriga, A.; Nillessen, B.; Kranzen, J.; Van Kessel, I. D. G.; Croes, H. J. E.; Aguilera, B.; De Visser, P. C.; Datson, N. A.; Mulders, S. A. M.; Van Deutekom, J. C. T.; et al. Intracellular Distribution and Nuclear Activity of Antisense Oligonucleotides After Unassisted Uptake in Myoblasts and Differentiated Myotubes In Vitro. Nucleic Acid Therapeutics 2017, 27 (3), 144-158. DOI: 10.1089/nat.2016.0641.
(16) Hung, G.; Xiao, X.; Peralta, R.; Bhattacharjee, G.; Murray, S.; Norris, D.; Guo, S.; Monia, B. P. Characterization of Target mRNA Reduction Through In Situ RNA Hybridization in Multiple Organ Systems Following Systemic Antisense Treatment in Animals. Nucleic Acid Therapeutics 2013, 23 (6), 369-378. DOI: 10.1089/nat.2013.0443.
(17) Geary, R. S.; Norris, D.; Yu, R.; Bennett, C. F. Pharmacokinetics, biodistribution and cell uptake of antisense oligonucleotides. Adv Drug Deliv Rev 2015, 87, 46-51. DOI: 10.1016/j.addr.2015.01.008.
(18) Pollak, A. J.; Zhao, L.; Vickers, T. A.; Huggins, I. J.; Liang, X.-H.; Stanley. Insights into innate immune activation via PS-ASO-protein-TLR9 interactions. Nucleic Acids Research 2022, 50 (14), 8107-8126. DOI: 10.1093/nar/gkac618.
(19) Alhamadani, F.; Zhang, K.; Parikh, R.; Wu, H.; Rasmussen, T. P.; Bahal, R.; Zhong, X.-B.; Manautou, J. E. Adverse Drug Reactions and Toxicity of the Food and Drug Administration-Approved Antisense Oligonucleotide Drugs. Drug Metab Dispos 2022, 50 (6), 879-887. DOI: 10.1124/dmd.121.000418.
(20) Crooke, S. T.; Vickers, T. A.; Liang, X.-H. Phosphorothioate modified oligonucleotide-protein interactions. Nucleic Acids Research 2020, 48 (10), 5235-5253. DOI: 10.1093/nar/gkaa299.
(21) Koller, E.; Vincent, T. M.; Chappell, A.; De, S.; Manoharan, M.; Bennett, C. F. Mechanisms of single-stranded phosphorothioate modified antisense oligonucleotide accumulation in hepatocytes. Nucleic Acids Research 2011, 39 (11), 4795-4807. DOI: 10.1093/nar/gkr089.
(22) Gagliardi, M.; Ashizawa, A. T. The Challenges and Strategies of Antisense Oligonucleotide Drug Delivery. Biomedicines 2021, 9 (4), 433. DOI: 10.3390/biomedicines9040433.
(23) Frieden, M.; Hansen, H. F.; Koch, T. Nuclease stability of LNA oligonucleotides and LNA-DNA chimeras. Nucleosides Nucleotides Nucleic Acids 2003, 22 (5-8), 1041-1043. DOI: 10.1081/NCN-120022731.
(24) Meier, C.; Engels, J. W. Peptide Nucleic Acids(PNAs)—Unusual Properties of Nonionic Oligonucleotide Analogues. Angewandte Chemie International Edition in English 1992, 31 (8), 1008-1010. DOI: 10.1002/anie.199210081.
(25) Hudziak, R. M.; Barofsky, E.; Barofsky, D. F.; Weller, D. L.; Huang, S. B.; Weller, D. D. Resistance of morpholino phosphorodiamidate oligomers to enzymatic degradation. Antisense Nucleic Acid Drug Dev 1996, 6 (4), 267-272. DOI: 10.1089/oli.1.1996.6.267.
(26) Dande, P.; Prakash, T. P.; Sioufi, N.; Gaus, H.; Jarres, R.; Berdeja, A.; Swayze, E. E.; Griffey, R. H.; Bhat, B. Improving RNA interference in mammalian cells by 4′-thio-modified small interfering RNA (siRNA): effect on siRNA activity and nuclease stability when used in combination with 2′-O-alkyl modifications. Journal of Medicinal Chemistry 2006, 49 (5), 1624-1634. DOI: 10.1021/jm050822c.
(27) Pascual-Gilabert, M.; Artero, R.; López-Castel, A. The myotonic dystrophy type 1 drug development pipeline: 2022 edition. Drug Discovery Today 2023, 28 (3), 103489. DOI: 10.1016/j.drudis.2023.103489.
(28) Klein, A. F.; Varela, M. A.; Arandel, L.; Holland, A.; Naouar, N.; Arzumanov, A.; Seoane, D.; Revillod, L.; Bassez, G.; Ferry, A.; et al. Peptide-conjugated oligonucleotides evoke long-lasting myotonic dystrophy correction in patient-derived cells and mice. J Clin Invest 2019, 129 (11), 4739-4744. DOI: 10.1172/JCI128205.
(29) Stoodley, J.; Miraz, D. S.; Jad, Y.; Fischer, M.; Wood, M. J. A.; Varela, M. A. Peptide-Conjugated PMOs for the Treatment of Myotonic Dystrophy. In Muscular Dystrophy Therapeutics: Methods and Protocols, Maruyama, R., Yokota, T. Eds.; Methods in Molecular Biology, Springer US, 2023; pp 209-237.
(30) Stoodley, J.; Vallejo-Bedia, F.; Seone-Miraz, D.; Debasa-Mouce, M.; Wood, M. J. A.; Varela, M. A. Application of Antisense Conjugates for the Treatment of Myotonic Dystrophy Type 1. International Journal of Molecular Sciences 2023, 24 (3), 2697. DOI: 10.3390/ijms24032697 (acccessed 2023/03/24/19:20:01). From www.mdpi.com.
(31) Copolovici, D. M.; Langel, K.; Eriste, E.; Langel, Ü. Cell-Penetrating Peptides: Design, Synthesis, and Applications. ACS Nano 2014, 8 (3), 1972-1994. DOI: 10.1021/nn4057269.
(32) Hansel, T. T.; Kropshofer, H.; Singer, T.; Mitchell, J. A.; George, A. J. T. The safety and side effects of monoclonal antibodies. Nature Reviews Drug Discovery 2010, 9 (4), 325-338. DOI: 10.1038/nrd3003.
(33) Candelaria, P. V.; Leoh, L. S.; Penichet, M. L.; Daniels-Wells, T. R. Antibodies Targeting the Transferrin Receptor 1 (TfR1) as Direct Anti-cancer Agents. Front. Immunol. 2021, 12. DOI: 10.3389/fimmu.2021.607692.
(34) Saar, K.; Lindgren, M.; Hansen, M.; Eiriksdóttir, E.; Jiang, Y.; Rosenthal-Aizman, K.; Sassian, M.; Langel, Ü. Cell-penetrating peptides: A comparative membrane toxicity study. Analytical Biochemistry 2005, 345 (1), 55-65. DOI: 10.1016/j.ab.2005.07.033.
(35) Wang, D.; Lin, J.; Jia, F.; Tan, X.; Wang, Y.; Sun, X.; Cao, X.; Che, F.; Lu, H.; Gao, X.; et al. Bottlebrush-architectured poly(ethylene glycol) as an efficient vector for RNA interference in vivo. Science Advances 2019, 5 (2), eaav9322. DOI: 10.1 126/sciadv.aav9322.
(36) Wang, Y.; Wang, D.; Jia, F.; Miller, A.; Tan, X.; Chen, P.; Zhang, L.; Lu, H.; Fang, Y.; Kang, X.; et al. Self-Assembled DNA-PEG Bottlebrushes Enhance Antisense Activity and Pharmacokinetics of Oligonucleotides. ACS Appl. Mater. Interfaces 2020, 12 (41), 45830-45837. DOI: 10.1021/acsami.0c13995.
(37) Zhang, L.; Wang, Y.; Chen, P.; Wang, D.; Sun, T.; Zhang, Z.; Wang, R.; Kang, X.; Fang, Y.; Lu, H.; et al. A mechanistic study on the cellular uptake, intracellular trafficking, and antisense gene regulation of bottlebrush polymer-conjugated oligonucleotides. RSC Chem. Biol. 2023, 4 (2), 138-145. DOI: 10.1039/D2CB00149G.
(38) Su, C.-F.; Merlitz, H.; Rabbel, H.; Sommer, J.-U. Nanoparticles of Various Degrees of Hydrophobicity Interacting with Lipid Membranes. J Phys Chem Lett 2017, 8 (17), 4069-4076. DOI: 10.1021/acs.jpclett.7b01888.
(39) Pearce, A. K.; O'Reilly, R. K. Polymers for Biomedical Applications: The Importance of Hydrophobicity in Directing Biological Interactions and Application Efficacy. Biomacromolecules 2021, 22 (11), 4459-4469. DOI: 10.1021/acs.biomac.1c00434.
(40) Jia, F.; Lu, X.; Tan, X.; Wang, D.; Cao, X.; Zhang, K. Effect of PEG Architecture on the Hybridization Thermodynamics and Protein Accessibility of PEGylated Oligonucleotides. Angew Chem Int Ed Engl 2017, 56 (5), 1239-1243. DOI: 10.1002/anie.201610753.
(41) Lu, X.; Jia, F.; Tan, X.; Wang, D.; Cao, X.; Zheng, J.; Zhang, K. Effective Antisense Gene Regulation via Noncationic, Polyethylene Glycol Brushes. J. Am. Chem. Soc. 2016, 138 (29), 9097-9100. DOI: 10.1021/jacs.6b05787.
(42) Lennox, K. A.; Behlke, M. A. Cellular localization of long non-coding RNAs affects silencing by RNAi more than by antisense oligonucleotides. Nucleic Acids Research 2016, 44 (2), 863-877. DOI: 10.1093/nar/gkv1206.
(43) Buntz, A.; Killian, T.; Schmid, D.; Seul, H.; Brinkmann, U.; Ravn, J.; Lindholm, M.; Knoetgen, H.; Haucke, V.; Mundigl, O. Quantitative fluorescence imaging determines the absolute number of locked nucleic acid oligonucleotides needed for suppression of target gene expression. Nucleic Acids Research 2019, 47 (2), 953-969. DOI: 10.1093/nar/gky1158.
(44) Roberts, J.; Palma, E.; Sazani, P.; Ørum, H.; Cho, M.; Kole, R. Efficient and Persistent Splice Switching by Systemically Delivered LNA Oligonucleotides in Mice. Mol Ther 2006, 14 (4), 471-475. DOI: 10.1016/j.ymthe.2006.05.017.
(45) Colwell, L. J.; Brenner Mp Fau—Ribbeck, K.; Ribbeck, K. Charge as a selection criterion for translocation through the nuclear pore complex. (1553-7358 (Electronic)). From 2010 Apr. 22.
(46) Lueck, J. D.; Lungu, C.; Mankodi, A.; Osborne, R. J.; Welle, S. L.; Dirksen, R. T.; Thornton, C. A. Chloride channelopathy in myotonic dystrophy resulting from loss of posttranscriptional regulation for CLCN1. American Journal of Physiology-Cell Physiology 2007, 292 (4), C1291-C1297. DOI: 10.1152/ajpcell.00336.2006.
(47) Santoro, M.; Piacentini, R.; Masciullo, M.; Bianchi, M. L. E.; Modoni, A.; Podda, M. V.; Ricci, E.; Silvestri, G.; Grassi, C. Alternative splicing alterations of Ca²⁺ handling genes are associated with Ca²⁺ signal dysregulation in myotonic dystrophy type 1 (DM1) and type 2 (DM2) myotubes. Neuropathology and Applied Neurobiology 2014, 40 (4), 464-476. DOI: 10.1111/nan.12076.
(48) Nitschke, L.; Cooper, T. A. Combinatorial effects of ion channel mis-splicing as a cause of myopathy in myotonic dystrophy. Journal of Clinical Investigation 2024, 134 (1). DOI: 10.1172/jci176089.
(49) Wheeler, T. M.; Lueck, J. D.; Swanson, M. S.; Dirksen, R. T.; Thornton, C. A. Correction of ClC-1 splicing eliminates chloride channelopathy and myotonia in mouse models of myotonic dystrophy. Journal of Clinical Investigation 2007. DOI: 10.1172/jci33355.
(50) Mankodi, A.; Takahashi, M. P.; Jiang, H.; Beck, C. L.; Bowers, W. J.; Moxley, R. T.; Cannon, S. C.; Thornton, C. A. Expanded CUG Repeats Trigger Aberrant Splicing of ClC-1 Chloride Channel Pre-mRNA and Hyperexcitability of Skeletal Muscle in Myotonic Dystrophy. Mol Cell 2002, 10 (1), 35-44. DOI: 10.1016/s1097-2765(02)00563-4.
(51) Osborne, R. J.; Lin, X.; Welle, S.; Sobczak, K.; O'Rourke, J. R.; Swanson, M. S.; Thornton, C. A. Transcriptional and post-transcriptional impact of toxic RNA in myotonic dystrophy. Human Molecular Genetics 2009, 18 (8), 1471-1481. DOI: 10.1093/hmg/ddp058.
(52) López-Martinez, A.; Soblechero-Martin, P.; de-la-Puente-Ovejero, L.; Nogales-Gadea, G.; Arechavala-Gomeza, V. An Overview of Alternative Splicing Defects Implicated in Myotonic Dystrophy Type I. Genes (Basel) 2020, 11 (9), 1109. DOI: 10.3390/genes11091109.
(53) Tanner, M. K.; Tang, Z.; Thornton, C. A. Targeted splice sequencing reveals RNA toxicity and therapeutic response in myotonic dystrophy. Nucleic Acids Research 2021, 49 (4), 2240-2254. DOI: 10.1093/nar/gkab022.
(54) Kim, E. Y.; Barefield, D. Y.; Vo, A. H.; Gacita, A. M.; Schuster, E. J.; Wyatt, E. J.; Davis, J. L.; Dong, B.; Sun, C.; Page, P.; et al. Distinct pathological signatures in human cellular models of myotonic dystrophy subtypes. JCI Insight 2019. DOI: 10.1172/jci.insight.122686.
(55) Solovyeva, E. M.; Utzinger, S.; Vissieres, A.; Mitchelmore, J.; Ahrnè, E.; Hermes, E.; Poetsch, T.; Ronco, M.; Bidinosti, M.; Merkl, C.; et al. Integrative Proteogenomics for Differential Expression and Splicing Variation in a DM1 Mouse Model. Molecular & Cellular Proteomics 2024, 23 (1), 100683. DOI: 10.1016/j.mcpro.2023.100683.
(56) Mateus, T.; Martins, F.; Nunes, A.; Herdeiro, M. T.; Rebelo, S. Metabolic Alterations in Myotonic Dystrophy Type 1 and Their Correlation with Lipin. International Journal of Environmental Research and Public Health 2021, 18 (4), 1794. DOI: 10.3390/ijerph18041794.
(57) Savkur, R. S.; Philips, A. V.; Cooper, T. A. Aberrant regulation of insulin receptor alternative splicing is associated with insulin resistance in myotonic dystrophy. Nature Genetics 2001, 29 (1), 40-47. DOI: 10.1038/ng704.
(58) Jearawiriyapaisarn, N.; Moulton, H. M.; Buckley, B.; Roberts, J.; Sazani, P.; Fucharoen, S.; Iversen, P. L.; Kole, R. Sustained Dystrophin Expression Induced by Peptide-conjugated Morpholino Oligomers in the Muscles of mdx Mice. Mol Ther 2008, 16 (9), 1624-1629. DOI: 10.1038/mt.2008.120.
(59) Fearnley, L. G.; Bennett, M. F.; Bahlo, M. Detection of repeat expansions in large next generation DNA and RNA sequencing data without alignment. Scientific Reports 2022, 12 (1), 13124. DOI: 10.1038/s41598-022-17267-z.
(60) Thornton, C. A.; Moxley, R. T.; Eichinger, K.; Heatwole, C.; Mignon, L.; Arnold, W. D.; Ashizawa, T.; Day, J. W.; Dent, G.; Tanner, M. K.; et al. Antisense oligonucleotide targeting DMPK in patients with myotonic dystrophy type 1: a multicentre, randomised, dose-escalation, placebo-controlled, phase 1/2a trial. The Lancet Neurology 2023, 22 (3), 218-228. DOI: 10.1016/s1474-4422(23)00001-7.
(61) Subramanian. Muscle Targeting Complexes and Uses Thereof for Treating Myotonic Dystrophy. United States 2021.
(62) Empowering Oligonucleotide Therapeutics. In Company Presentation, March 2023, PepGen: 2023.
(63) Yiming Zhu, T. K., Qingying Meng, Li-Jung Tai, Michelle Lee, Husam Younis, Art Levin, Mike Flanagan. AOC 1001 Demonstrates DMPK Reduction and Spliceopathy Improvement in a Phase 1/2 Study in Myotonic Dystrophy Type 1 (DM1) (MARINA™) Avidity Biosciences Inc.: 28th Annual Congress of the World Muscle Society, Charleston, SC, 2023.
(64) Therapeutics, D. Achieving the Promise of FORCE TO Deliver for Patients. In Achieve & Deliver Clinical Data Update, Jan. 3, 2024, 2024.
(65) Nitin Nitin, G. B. NLS Peptide Conjugated Molecular Beacons for Visualizing Nuclear RNA in Living Cells. Bioconj Chem 2008, 19 (11), 2205-2211. DOI: 10.1021/bc800322a.
(66) Tammam, S. N.; Azzazy, H. M. E.; Lamprecht, A. The effect of nanoparticle size and NLS density on nuclear targeting in cancer and normal cells; impaired nuclear import and aberrant nanoparticle intracellular trafficking in glioma. Journal of Controlled Release 2017, 253, 30-36. DOI: 10.1016/j.jconrel.2017.02.029.
(67) McQuaid, C.; Halsey, A.; Dubois, M.; Romero, I.; Male, D. Comparison of polypeptides that bind the transferrin receptor for targeting gold nanocarriers. PLoS One 2021, 16 (6), e0252341. DOI: 10.1371/journal.pone.0252341.
(68) Crook, Z. R.; Girard, E.; Sevilla, G. P.; Merrill, M.; Friend, D.; Rupert, P. B.; Pakiam, F.; Nguyen, E.; Yin, C.; Ruff, R. O.; et al. A TfR-Binding Cystine-Dense Peptide Promotes Blood-Brain Barrier Penetration of Bioactive Molecules. Journal of Molecular Biology 2020, 432 (14), 3989-4009. DOI: 10.1016/j.jmb.2020.04.002.
(69) Sun, P.; Wu, Z.; Xiao, Y.; Wu, H.; Di, Q.; Zhao, X.; Quan, J.; Tang, H.; Wang, Q.; Chen, W. TfR-T12 short peptide and pH sensitive cell transmembrane peptide modified nano-composite micelles for glioma treatment via remodeling tumor microenvironment. Nanomedicine: Nanotechnology, Biology and Medicine 2022, 41, 102516. DOI: 10.1016/j.nano.2022.102516.
(70) Li, W.; Szoka, F. C. Lipid-based Nanoparticles for Nucleic Acid Delivery. Pharm Res 2007, 24 (3), 438-449. DOI: 10.1007/s11095-006-9180-5.
(71) de Mezer, M.; Wojciechowska, M.; Napierala, M.; Sobczak, K.; Krzyzosiak, W. J. Mutant CAG repeats of Huntingtin transcript fold into hairpins, form nuclear foci and are targets for RNA interference. Nucleic Acids Research 2011, 39 (9), 3852-3863. DOI: 10.1093/nar/gkq1323.
(72) Lu, X.; Watts, E.; Jia, F.; Tan, X.; Zhang, K. Polycondensation of Polymer Brushes via DNA Hybridization. J. Am. Chem. Soc. 2014, 136 (29), 10214-10217. DOI: 10.1021/ja504790r (accessed 2023/04/26/20:12:24).
(73) Forcina, V.; Garcia-Dominguez, A.; Lloyd-Jones, G. C. Kinetics of initiation of the third generation Grubbs metathesis catalyst: convergent associative and dissociative pathways. Faraday Discussions 2019, 220, 179-195. DOI: 10.1039/c9fd00043g.
(74) Shen, S.; Park, J. W.; Lu, Z.-x.; Lin, L.; Henry, M. D.; Wu, Y. N.; Zhou, Q.; Xing, Y. rMATS: Robust and flexible detection of differential alternative splicing from replicate RNA-Seq data. Proc. Natl. Acad. Sci. U.S.A. 2014, 111 (51), E5593-E5601. DOI: 10.1073/pnas.1419161111.

The teachings of all manuscripts, patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method for treating muscular dystrophy in a subject in need thereof, comprising administering to the subject a therapeutically effective amount of a composition comprising a bottlebrush polymer-oligonucleotide conjugate.

2. The method of claim 1, wherein the subject has muscular dystrophy selected from Duchenne muscular dystrophy (DMD), myotonic dystrophy type 1 (DM1), and facioscapulohumeral muscular dystrophy (FSHD).

3. The method of claim 1, wherein the bottlebrush polymer-oligonucleotide conjugate comprises:
a polymer backbone;
polyethylene glycol (PEG) polymer arms covalently linked to the polymer backbone; and
an oligonucleotide covalently linked to the backbone.

4. The method of claim 3, wherein the oligonucleotide comprises:
a single stranded nucleic acid;
a double stranded nucleic acid;
a chemically modified nucleic acid; or
any combination of the foregoing.

5. The method of claim 3, wherein the oligonucleotide comprises:
one or more locked nucleic acid (LNA) modified bases;
an antisense oligonucleotide (ASO);
a small interfering RNA (siRNA);
a sequence complementary to a region of a pathogenic transcript; or
any combination of the foregoing.

6. The method of claim 5, wherein the pathogenic transcript comprises:
a human DM1 protein kinase (DMPK) transcript containing CUG repeats;
a mutated human dystrophin (DMD) transcript; or
a human double homeobox 4 (DUX4) transcript.

7. The method of claim 5, wherein the oligonucleotide comprises a nucleic acid sequence of 5′-CAGCAGCAG-3′.

8. The method of claim 3, wherein the polymer backbone comprises one or more monomers.

9. The method of claim 8, wherein at least one of the monomers is selected from a synthetic monomer, a natural monomer, and a modified form of a natural molecule.

10. The method of claim 9, wherein:
the synthetic monomer is selected from serinol, norbornene, acrylate, and acrylamide;
the natural monomer is selected from amino acid and sugar;
the modified form of a natural molecule is selected from a morpholino phosphorodiamidate, a modified amino acid, a modified spermine, a modified lipid, and a modified cholesterol;
or any combination of the foregoing.

11. The method of claim 3, wherein:
the bottlebrush polymer-oligonucleotide conjugate comprises about 15 to about 30 PEG polymer arms;
each of the PEG polymer arms is about 2 kDa to about 20 kDa;
or both of the foregoing.

12. The method of claim 3, wherein:
the bottlebrush polymer-oligonucleotide conjugate comprises about 30 PEG polymer arms;
each of the PEG polymer arms is about 10 kDa;
or both of the foregoing.

13. The method of claim 1, wherein the bottlebrush polymer-oligonucleotide conjugate further comprises a radiochemical label, a targeting ligand, or both.

14. The method of 13, wherein the targeting ligand comprises a nuclear localization sequence (NLS) peptide, a transferrin receptor 1-targeting peptide, or a pH-sensitive lipid.

15. The method of claim 1, wherein administering the bottlebrush polymer-oligonucleotide conjugate to the subject induces alternative splicing.

16. The method of claim 1, wherein the composition is administered to the subject:
as a dose of about 1 mg oligonucleotide per kg bodyweight (1 mg/kg) to about 80 mg oligonucleotide per kg bodyweight (80 mg/kg);
by intravenous injection, intramuscular injection, intraperitoneal injection, or subcutaneous injection; or
any combination of the foregoing.

17. A bottlebrush polymer-oligonucleotide conjugate, comprising:
a polymer backbone;
polyethylene glycol (PEG) polymer arms covalently linked to the polymer backbone; and
an oligonucleotide covalently linked to the backbone, wherein the oligonucleotide comprises a sequence complementary to a region of a pathogenic transcript associated with a muscular dystrophy.

18. A composition comprising the bottlebrush polymer-oligonucleotide conjugate of claim 17.

19. A method of producing the bottlebrush polymer-oligonucleotide conjugate of claim 17, comprising:
synthesizing a bottlebrush polymer; and
conjugating the bottlebrush polymer with an oligonucleotide comprising a sequence complementary to a region of a pathogenic transcript associated with a muscular dystrophy, thereby producing the bottlebrush polymer-oligonucleotide conjugate.

20. The method of claim 19, wherein:
synthesizing the bottlebrush polymer comprises polymerizing norbornenyl bromide and norbornenyl PEG; and
conjugating the bottlebrush polymer with the oligonucleotide comprises:
conjugating the bottlebrush polymer with an azide group, thereby producing an azide-functionalized bottlebrush polymer; and
reacting the azide-functionalized bottlebrush polymer and the oligonucleotide, wherein the oligonucleotide is a DBCO-modified oligonucleotide, thereby producing the bottlebrush polymer-oligonucleotide conjugate.
